SEQUENCE LISTING (174 sequences)

SEQ ID NO: 1 - RNA, 36 nt, synthetic construct
  source 1..36: mol_type = other RNA
nnnnnnnnnnnnnnnnnnnngttttagagctatgct

SEQ ID NO: 2 - RNA, 67 nt, synthetic construct
  source 1..67: mol_type = other RNA
agcatagcaagttaaaataaggctagtccgttatcaacttgaaaaagtggcaccgagtcggtgcttt

SEQ ID NO: 3 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,20,22..25,33..36): mod_base = OTHER; note = Phosphorothioate linkages

SEQ ID NO: 4 - RNA, 36 nt, synthetic construct
  source 1..36: mol_type = other RNA
gagacaaatcacctgcctcggttttagagctatgct

SEQ ID NO: 5 - RNA, 36 nt, synthetic construct; identical to SEQ ID NO: 1
  source 1..36: mol_type = other RNA
  modified_base order(1..10,16..18,20,21,25..36): mod_base = OTHER; note = 2-Prime-O-methyl RNA nucleotides
  modified_base order(1..4,33..35): mod_base = OTHER; note = Phosphorothioate linkages

SEQ ID NO: 6 - RNA, 36 nt, synthetic construct; identical to SEQ ID NO: 1
  source 1..36: mol_type = other RNA
  modified_base order(7..10,20,21,27..36): mod_base = OTHER; note = 2-Prime-O-methyl RNA nucleotides
  modified_base 33..35: mod_base = OTHER; note = Phosphorothioate linkages

SEQ ID NO: 7 - RNA, 36 nt, synthetic construct; identical to SEQ ID NO: 1
  source 1..36: mol_type = other RNA
  modified_base order(7..10,20,21,27..36): mod_base = OTHER; note = 2-Prime-O-methyl RNA nucleotides
  modified_base order(1..4,33..35): mod_base = OTHER; note = Phosphorothioate linkages

SEQ ID NO: 8 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(1..4,33..36): mod_base = OTHER; note = Phosphorothioate linkages

SEQ ID NO: 9 - RNA, 36 nt, synthetic construct; identical to SEQ ID NO: 1
  source 1..36: mol_type = other RNA
  modified_base order(7..10,17,18,20,21,27..36): mod_base = OTHER; note = 2-Prime-O-methyl RNA nucleotides
  modified_base order(1..4,33..36): mod_base = OTHER; note = Phosphorothioate linkages

SEQ ID NO: 10 - RNA, 36 nt, synthetic construct; identical to SEQ ID NO: 1
  source 1..36: mol_type = other RNA
  modified_base order(7..10,20,21,25..36): mod_base = OTHER; note = 2-Prime-O-methyl RNA nucleotides
  modified_base order(1..4,33..36): mod_base = OTHER; note = Phosphorothioate linkages

SEQ ID NO: 11 - RNA, 36 nt, synthetic construct; identical to SEQ ID NO: 1
  source 1..36: mol_type = other RNA
  modified_base order(1..10,17,18,20,21,27..36): mod_base = OTHER; note = 2-Prime-O-methyl RNA nucleotides
  modified_base order(1..4,33..36): mod_base = OTHER; note = Phosphorothioate linkages

SEQ ID NO: 12 - RNA, 36 nt, synthetic construct; identical to SEQ ID NO: 1
  source 1..36: mol_type = other RNA
  modified_base order(1..10,20,21,25..36): mod_base = OTHER; note = 2-Prime-O-methyl RNA nucleotides
  modified_base order(1..4,33..36): mod_base = OTHER; note = Phosphorothioate linkages

SEQ ID NO: 13 - RNA, 36 nt, synthetic construct; identical to SEQ ID NO: 1
  source 1..36: mol_type = other RNA
  modified_base order(1..10,20,21,25..36): mod_base = OTHER; note = 2-Prime-O-methyl RNA nucleotides
  modified_base order(1..4,15..17,19,20,22..25,33..36): mod_base = OTHER; note = Phosphorothioate linkages

SEQ ID NO: 14 - RNA, 36 nt, synthetic construct; identical to SEQ ID NO: 1
  source 1..36: mol_type = other RNA
  modified_base order(1..10,20,21,25..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..4,15..17,19,20,22..25,33..36): mod_base = OTHER; note = Phosphorothioate linkages

SEQ ID NO: 15 - RNA, 36 nt, synthetic construct; identical to SEQ ID NO: 1
  source 1..36: mol_type = other RNA
  modified_base order(1..10,20,21,25..36): mod_base = OTHER; note = 2-Prime-O-methyl RNA nucleotides
  modified_base 11..14: mod_base = OTHER; note = 2-Prime-fluoro RNA nucleotides
  modified_base order(1..4,15..20,22..25,33..36): mod_base = OTHER; note = Phosphorothioate linkages

SEQ ID NO: 16 - RNA, 36 nt, synthetic construct; identical to SEQ ID NO: 1
  source 1..36: mol_type = other RNA
  modified_base order(1..10,20,21,25,27..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..4,15..17,19,20,22..25,26,27,33..36): mod_base = OTHER; note = Phosphorothioate linkages

SEQ ID NO: 17 - RNA, 36 nt, synthetic construct; identical to SEQ ID NO: 1
  source 1..36: mol_type = other RNA
  modified_base order(1..10,20,21,26..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..4,15..17,19,20,22..26,33..36): mod_base = OTHER; note = Phosphorothioate linkages

SEQ ID NO: 18 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..4,15..17,19,20,22..27,33..36): mod_base = OTHER; note = Phosphorothioate linkages

SEQ ID NO: 19 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..4,33..36): mod_base = OTHER; note = Phosphorothioate linkages

SEQ ID NO: 20 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,22,24..26): mod_base = OTHER; note = 2-Prime-fluoro RNA nucleotides
  modified_base order(1..4,15..17,19,20,23,24,33..36): mod_base = OTHER; note = Phosphorothioate linkages

SEQ ID NO: 21 - RNA, 36 nt, synthetic construct; identical to SEQ ID NO: 1
  source 1..36: mol_type = other RNA
  modified_base order(1..10,15,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,16,17,19,20,22..25,33..36): mod_base = OTHER; note = Phosphorothioate linkages

SEQ ID NO: 22 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,16..18,25,26): mod_base = OTHER; note = 2-Prime-fluoro RNA nucleotides
  modified_base order(1..4,15,16,19,20,22..25,33..36): mod_base = OTHER; note = Phosphorothioate linkages

SEQ ID NO: 23 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17..19,25,26): mod_base = OTHER; note = 2-Prime-fluoro RNA nucleotides
  modified_base order(1..4,15..17,22..25,33..36): mod_base = OTHER; note = Phosphorothioate linkages

SEQ ID NO: 24 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,22,25,26): mod_base = OTHER; note = 2-Prime-fluoro RNA nucleotides
  modified_base order(1..4,15..17,19,20,23..25,33..36): mod_base = OTHER; note = Phosphorothioate linkages

SEQ ID NO: 25 - RNA, 37 nt, synthetic construct
  source 1..37: mol_type = other RNA
  modified_base order(1..10,22,28..37): mod_base = OTHER; note = 2-Prime-O-methyl RNA nucleotides
  modified_base order(11..14,17,18,24,26,27): mod_base = OTHER; note = 2-Prime-fluoro RNA nucleotides
  modified_base order(1..4,15..17,19,20,23,24,25,26,34..37): mod_base = OTHER; note = Phosphorothioate linkages
nnnnnnnnnnnnnnnnnnnnagttttagagctatgct

SEQ ID NO: 26 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,20,22..24,33..36): mod_base = OTHER; note = Phosphorothioate linkages

SEQ ID NO: 27 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,16..19,22..26): mod_base = OTHER; note = 2-Prime-fluoro RNA nucleotides
  modified_base order(1..4,15,16,33..36): mod_base = OTHER; note = Phosphorothioate linkages

SEQ ID NO: 28 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(1..4,33..36): mod_base = OTHER; note = Phosphorothioate linkages

SEQ ID NO: 29 - RNA, 36 nt, synthetic construct; identical to SEQ ID NO: 1
  source 1..36: mol_type = other RNA
  modified_base order(1..3,7..11,20,21,27..36): mod_base = OTHER; note = 2-Prime-O-methyl RNA nucleotides
  modified_base order(1..4,33..36): mod_base = OTHER; note = Phosphorothioate linkages

SEQ ID NO: 30 - RNA, 36 nt, synthetic construct; identical to SEQ ID NO: 1
  source 1..36: mol_type = other RNA
  modified_base order(1..3,7..11,13,14,20,21,27..36): mod_base = OTHER; note = 2-Prime-O-methyl RNA nucleotides
  modified_base order(1..4,33..36): mod_base = OTHER; note = Phosphorothioate linkages

SEQ ID NO: 31 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(1..7,11..20,22..27,33..36): mod_base = OTHER; note = Phosphorothioate linkages

SEQ ID NO: 32 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(1..7,11..20,33..36): mod_base = OTHER; note = Phosphorothioate linkages

SEQ ID NO: 33 - RNA, 36 nt, synthetic construct; identical to SEQ ID NO: 1
  source 1..36: mol_type = other RNA
  modified_base order(1..3,7..10,20,21,25..36): mod_base = OTHER; note = 2-Prime-O-methyl RNA nucleotides
  modified_base order(1..4,33..36): mod_base = OTHER; note = Phosphorothioate linkages

SEQ ID NO: 34 - RNA, 36 nt, synthetic construct; identical to SEQ ID NO: 1
  source 1..36: mol_type = other RNA
  modified_base order(1..3,7..10,20,21,25..36): mod_base = OTHER; note = 2-Prime-O-methyl RNA nucleotides
  modified_base order(1..7,11..20,33..36): mod_base = OTHER; note = Phosphorothioate linkages

SEQ ID NO: 35 - RNA, 36 nt, synthetic construct; identical to SEQ ID NO: 1
  source 1..36: mol_type = other RNA
  modified_base order(1..3,7..10,20,21,25..36): mod_base = OTHER; note = 2-Prime-O-methyl RNA nucleotides
  modified_base order(11..14,17,18): mod_base = OTHER; note = 2-Prime-fluoro RNA nucleotides
  modified_base order(1..7,15..17,19,20,22..25,33..36): mod_base = OTHER; note = Phosphorothioate linkages

SEQ ID NO: 36 - RNA, 36 nt, synthetic construct; identical to SEQ ID NO: 1
  source 1..36: mol_type = other RNA
  modified_base order(1..3,7..10,20,21,25..36): mod_base = OTHER; note = 2-Prime-O-methyl RNA nucleotides
  modified_base 4..6: mod_base = OTHER; note = 2-Prime-deoxy RNA nucleotides
  modified_base order(11..14,17,18): mod_base = OTHER; note = 2-Prime-fluoro RNA nucleotides
  modified_base order(1..7,15..17,19,20,22..25,33..36): mod_base = OTHER; note = Phosphorothioate linkages

SEQ ID NO: 37 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,33..36): mod_base = OTHER; note = Phosphorothioate linkages

SEQ ID NO: 38 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,33..36): mod_base = OTHER; note = Phosphorothioate linkages

SEQ ID NO: 39 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(15,16,19): mod_base = OTHER; note = 2-Prime-deoxy RNA nucleotides
  modified_base order(1..4,15..17,19,20,22..25,33..36): mod_base = OTHER; note = Phosphorothioate linkages

SEQ ID NO: 40 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 22..24: mod_base = OTHER; note = 2-Prime-deoxy RNA nucleotides
  modified_base order(1..4,15..17,19,20,22..25,33..36): mod_base = OTHER; note = Phosphorothioate linkages

SEQ ID NO: 41 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11,13,14,17,18,25,26): mod_base = OTHER; note = 2-Prime-fluoro RNA nucleotides
  modified_base order(1..4,12,13,15..17,19,20,22..25,33..36): mod_base = OTHER; note = Phosphorothioate linkages

SEQ ID NO: 42 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11,13,14,17,18,25,26): mod_base = OTHER; note = 2-Prime-fluoro RNA nucleotides
  modified_base 12: mod_base = OTHER; note = 2-Prime-deoxy RNA nucleotide
  modified_base order(1..4,12,13,15..17,19,20,22..25,33..36): mod_base = OTHER; note = Phosphorothioate linkages

SEQ ID NO: 43 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(4..6,11..19,22..26): mod_base = OTHER; note = 2-Prime-fluoro RNA nucleotides
  modified_base order(1..4,33..36): mod_base = OTHER; note = Phosphorothioate linkages

SEQ ID NO: 44 - RNA, 36 nt, synthetic construct; identical to SEQ ID NO: 1
  source 1..36: mol_type = other RNA
  modified_base order(1..10,15,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,16,17,19,20,22,23,24,25,33..36): mod_base = OTHER; note = Phosphorothioate linkages

SEQ ID NO: 45 - RNA, 36 nt, synthetic construct; identical to SEQ ID NO: 1
  source 1..36: mol_type = other RNA
  modified_base order(1..10,16,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15,16,19,20,22,23,24,25,33..36): mod_base = OTHER; note = Phosphorothioate linkages

SEQ ID NO: 46 - RNA, 36 nt, synthetic construct; identical to SEQ ID NO: 1
  source 1..36: mol_type = other RNA
  modified_base order(1..10,16,19..21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15,16,22,23,24,25,33..36): mod_base = OTHER; note = Phosphorothioate linkages

SEQ ID NO: 47 - RNA, 36 nt, synthetic construct; identical to SEQ ID NO: 1
  source 1..36: mol_type = other RNA
  modified_base order(1..10,20..22,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,20,23..25,33..36): mod_base = OTHER; note = Phosphorothioate linkages

SEQ ID NO: 48 - RNA, 36 nt, synthetic construct; identical to SEQ ID NO: 1
  source 1..36: mol_type = other RNA
  modified_base order(1..10,20,21,23,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,20,22,23,24,25,33..36): mod_base = OTHER; note = Phosphorothioate linkages

SEQ ID NO: 49 - RNA, 36 nt, synthetic construct; identical to SEQ ID NO: 1
  source 1..36: mol_type = other RNA
  modified_base order(1..10,20,21,24,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,20,22..24,33..36): mod_base = OTHER; note = Phosphorothioate linkages

SEQ ID NO: 50 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,15..17,19,20,22..27,33..36): mod_base = OTHER; note = Phosphorothioate linkages

SEQ ID NO: 51 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..8,33..36): mod_base = OTHER; note = Phosphorothioate linkages

SEQ ID NO: 52 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,8,9,33..36): mod_base = OTHER; note = Phosphorothioate linkages

SEQ ID NO: 53 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,9,10,33..36): mod_base = OTHER; note = Phosphorothioate linkages

SEQ ID NO: 54 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,10,11,33..36): mod_base = OTHER; note = Phosphorothioate linkages

SEQ ID NO: 55 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,11,12,33..36): mod_base = OTHER; note = Phosphorothioate linkages

SEQ ID NO: 56 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,12,13,33..36): mod_base = OTHER; note = Phosphorothioate linkages

SEQ ID NO: 57 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,13,14,33..36): mod_base = OTHER; note = Phosphorothioate linkages

SEQ ID NO: 58 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,14,15,33..36): mod_base = OTHER; note = Phosphorothioate linkages

SEQ ID NO: 59 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,17,18,33..36): mod_base = OTHER; note = Phosphorothioate linkages

SEQ ID NO: 60 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,18,19,33..36): mod_base = OTHER; note = Phosphorothioate linkages

SEQ ID NO: 61 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,20,21,33..36): mod_base = OTHER; note = Phosphorothioate linkages

SEQ ID NO: 62 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,21,22,33..36): mod_base = OTHER; note = Phosphorothioate linkages

SEQ ID NO: 63 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,27,28,33..36): mod_base = OTHER; note = Phosphorothioate linkages

SEQ ID NO: 64 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,28,29,33..36): mod_base = OTHER; note = Phosphorothioate linkages

SEQ ID NO: 65 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,29,30,33..36): mod_base = OTHER; note = Phosphorothioate linkages

SEQ ID NO: 66 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,30,31,33..36): mod_base = OTHER; note = Phosphorothioate linkages

SEQ ID NO: 67 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,31,32,33..36): mod_base = OTHER; note = Phosphorothioate linkages

SEQ ID NO: 68 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,32..36): mod_base = OTHER; note = Phosphorothioate linkages

SEQ ID NO: 69 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..10,15..17,19,20,22..27,33..36): mod_base = OTHER; note = Phosphorothioate linkages

SEQ ID NO: 70 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,10..13,15..17,19,20,22..27,33..36): mod_base = OTHER; note = Phosphorothioate linkages

SEQ ID NO: 71 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,13..17,19,20,22..27,33..36): mod_base = OTHER; note = Phosphorothioate linkages

SEQ ID NO: 72 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,15..20,22..27,33..36): mod_base = OTHER; note = Phosphorothioate linkages

SEQ ID NO: 73 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,15..17,19..21,22..27,33..36): mod_base = OTHER; note = Phosphorothioate linkages

SEQ ID NO: 74 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..10,33..36): mod_base = OTHER; note = Phosphorothioate linkages

SEQ ID NO: 75 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..13,33..36): mod_base = OTHER; note = Phosphorothioate linkages

SEQ ID NO: 76 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..17,33..36): mod_base = OTHER; note = Phosphorothioate linkages

SEQ ID NO: 77 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..20,33..36): mod_base = OTHER; note = Phosphorothioate linkages

SEQ ID NO: 78 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..27,33..36): mod_base = OTHER; note = Phosphorothioate linkages

SEQ ID NO: 79 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,15..17,19,20,22..27,30..36): mod_base = OTHER; note = Phosphorothioate linkages

SEQ ID NO: 80 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,15..17,19,20,22..30,33..36): mod_base = OTHER; note = Phosphorothioate linkages

SEQ ID NO: 81 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,15..17,19,20,21..27,33..36): mod_base = OTHER; note = Phosphorothioate linkages

SEQ ID NO: 82 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,30..36): mod_base = OTHER; note = Phosphorothioate linkages

SEQ ID NO: 83 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,26..36): mod_base = OTHER; note = Phosphorothioate linkages

SEQ ID NO: 84 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..7,22..36): mod_base = OTHER; note = Phosphorothioate linkages

SEQ ID NO: 85 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..9,31..36): mod_base = OTHER; note = Phosphorothioate linkages

SEQ ID NO: 86 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..11,29..36): mod_base = OTHER; note = Phosphorothioate linkages

SEQ ID NO: 87 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..13,27..36): mod_base = OTHER; note = Phosphorothioate linkages

SEQ ID NO: 88 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..15,25..36): mod_base = OTHER; note = Phosphorothioate linkages

SEQ ID NO: 89 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..17,23..36): mod_base = OTHER; note = Phosphorothioate linkages

SEQ ID NO: 90 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base order(1..20,22..36): mod_base = OTHER; note = Phosphorothioate linkages

SEQ ID NO: 91 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base 4..6: mod_base = OTHER; note = 2-Prime-deoxy RNA nucleotides
  modified_base order(11..19,22..26): mod_base = OTHER; note = 2-Prime-fluoro RNA nucleotides
  modified_base 1..36: mod_base = OTHER; note = Phosphorothioate linkages

SEQ ID NO: 92 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,22..25,33..36): mod_base = OTHER; note = Phosphorothioate linkages

SEQ ID NO: 93 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..8,15..17,19,20,22..25,33..36): mod_base = OTHER; note = Phosphorothioate linkages

SEQ ID NO: 94 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,22..25,30,31,33..36): mod_base = OTHER; note = Phosphorothioate linkages

SEQ ID NO: 95 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,22..25,31,32,33..36): mod_base = OTHER; note = Phosphorothioate linkages

SEQ ID NO: 96 - RNA, 37 nt, synthetic construct
  source 1..37: mol_type = other RNA
  modified_base order(1..10,20,22,28..37): mod_base = OTHER; note = 2-Prime-O-methyl RNA nucleotides
  modified_base order(11..14,17,18,26,27): mod_base = OTHER; note = 2-Prime-fluoro RNA nucleotides
  modified_base order(1..7,15..17,19,20,23..26,33..37): mod_base = OTHER; note = Phosphorothioate linkages
nnnnnnnnnnnnnnnnnnngngttttagagctatgct

SEQ ID NO: 97 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,22..27,33..36): mod_base = OTHER; note = Phosphorothioate linkages

SEQ ID NO: 98 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,22..27,30..36): mod_base = OTHER; note = Phosphorothioate linkages

SEQ ID NO: 99 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,22..30,33..36): mod_base = OTHER; note = Phosphorothioate linkages

SEQ ID NO: 100 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,21..27,33..36): mod_base = OTHER; note = Phosphorothioate linkages

SEQ ID NO: 101 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,22..25,30..36): mod_base = OTHER; note = Phosphorothioate linkages

SEQ ID NO: 102 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..7,15..17,19,20,22..25,26..36): mod_base = OTHER; note = Phosphorothioate linkages

SEQ ID NO: 103 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..8,33..36): mod_base = OTHER; note = Phosphorothioate linkages

SEQ ID NO: 104 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,32..36): mod_base = OTHER; note = Phosphorothioate linkages

SEQ ID NO: 105 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..20,22..27,33..36): mod_base = OTHER; note = Phosphorothioate linkages

SEQ ID NO: 106 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..17,19,20,22..27,30..36): mod_base = OTHER; note = Phosphorothioate linkages

SEQ ID NO: 107 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..17,19,20,22..30,33..36): mod_base = OTHER; note = Phosphorothioate linkages

SEQ ID NO: 108 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,15..17,19,20,21..27,33..36): mod_base = OTHER; note = Phosphorothioate linkages

SEQ ID NO: 109 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,30..32,33..36): mod_base = OTHER; note = Phosphorothioate linkages

SEQ ID NO: 110 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(11..19,22..26): mod_base = OTHER; note = 2-Prime-fluoro RNA nucleotides
  modified_base order(1..7,26..32,33..36): mod_base = OTHER; note = Phosphorothioate linkages

SEQ ID NO: 111 - RNA, 36 nt, synthetic construct; identical to SEQ ID NO: 1
  source 1..36: mol_type = other RNA
  modified_base order(1..3,7..10,20,21,27..36): mod_base = OTHER; note = 2-Prime-O-methyl RNA nucleotides
  modified_base order(4..6,15,16,19,22..24): mod_base = OTHER; note = 2-Prime-deoxy RNA nucleotides
  modified_base order(11..14,17,18,25,26): mod_base = OTHER; note = 2-Prime-fluoro RNA nucleotides
  modified_base order(1..7,15..17,19,20,22..25,33..36): mod_base = OTHER; note = Phosphorothioate linkages

SEQ ID NO: 112 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(19,22..24): mod_base = OTHER; note = 2-Prime-NH2 (2-Prime-amino RNA) nucleotides
  modified_base order(1..4,15..17,33..36): mod_base = OTHER; note = Phosphorothioate linkages

SEQ ID NO: 113 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 19: mod_base = OTHER; note = 2-Prime-NH2 (2-Prime-amino RNA) nucleotide
  modified_base order(1..4,15..17,22..25,33..36): mod_base = OTHER; note = Phosphorothioate linkages

SEQ ID NO: 114 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 22: mod_base = OTHER; note = 2-Prime-NH2 (2-Prime-amino RNA) nucleotide
  modified_base order(1..4,15..17,19,20,23..25,33..36): mod_base = OTHER; note = Phosphorothioate linkages

SEQ ID NO: 115 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 23: mod_base = OTHER; note = 2-Prime-NH2 (2-Prime-amino RNA) nucleotide
  modified_base order(1..4,15..17,19,20,22,23,24,25,33..36): mod_base = OTHER; note = Phosphorothioate linkages

SEQ ID NO: 116 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 24: mod_base = OTHER; note = 2-Prime-NH2 (2-Prime-amino RNA) nucleotide
  modified_base order(1..4,15..17,19,20,22..24,33..36): mod_base = OTHER; note = Phosphorothioate linkages

SEQ ID NO: 117 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(19,22..24): mod_base = OTHER; note = 4-Prime-thio RNA nucleotides
  modified_base order(1..4,15..17,19,20,22..25,33..36): mod_base = OTHER; note = Phosphorothioate linkages

SEQ ID NO: 118 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(19,22..24): mod_base = OTHER; note = 4-Prime-thio RNA nucleotides
  modified_base order(1..4,15..17,33..36): mod_base = OTHER; note = Phosphorothioate linkages

SEQ ID NO: 119 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 19: mod_base = OTHER; note = 4-Prime-thio RNA nucleotide
  modified_base order(1..4,15..17,22..25,33..36): mod_base = OTHER; note = Phosphorothioate linkages

SEQ ID NO: 120 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 22: mod_base = OTHER; note = 4-Prime-thio RNA nucleotide
  modified_base order(1..4,15..17,19,20,23..25,33..36): mod_base = OTHER; note = Phosphorothioate linkages

SEQ ID NO: 121 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 23: mod_base = OTHER; note = 4-Prime-thio RNA nucleotide
  modified_base order(1..4,15..17,19,20,22,23,24,25,33..36): mod_base = OTHER; note = Phosphorothioate linkages

SEQ ID NO: 122 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 24: mod_base = OTHER; note = 4-Prime-thio RNA nucleotide
  modified_base order(1..4,15..17,19,20,22..24,33..36): mod_base = OTHER; note = Phosphorothioate linkages

SEQ ID NO: 123 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(22,24): mod_base = OTHER; note = 4-Prime-thio RNA nucleotides
  modified_base order(1..4,15..17,19,20,23,24,33..36): mod_base = OTHER; note = Phosphorothioate linkages

SEQ ID NO: 124 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 22..23: mod_base = OTHER; note = 4-Prime-thio RNA nucleotides
  modified_base order(1..4,15..17,19,20,24,25,33..36): mod_base = OTHER; note = Phosphorothioate linkages

SEQ ID NO: 125 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 23..24: mod_base = OTHER; note = 4-Prime-thio RNA nucleotides
  modified_base order(1..4,15..17,19,20,22,23,33..36): mod_base = OTHER; note = Phosphorothioate linkages

SEQ ID NO: 126 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 22..24: mod_base = OTHER; note = 2-Prime-NH2 (2-Prime-amino RNA) nucleotides
  modified_base order(1..4,15..17,19,20,33..36): mod_base = OTHER; note = Phosphorothioate linkages

SEQ ID NO: 127 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 22..24: mod_base = OTHER; note = 4-Prime-thio RNA nucleotides
  modified_base order(1..4,15..17,19,20,22..25,33..36): mod_base = OTHER; note = Phosphorothioate linkages

SEQ ID NO: 128 - RNA, 36 nt, synthetic construct; identical to SEQ ID NO: 1
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 22..24: mod_base = OTHER; note = 4-Prime-thio RNA nucleotides
  modified_base order(1..4,15..17,19,20,33..36): mod_base = OTHER; note = Phosphorothioate linkages

SEQ ID NO: 129 - RNA, 37 nt, synthetic construct
  source 1..37: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base 16: mod_base = OTHER; note = 2-Prime-NH2 (2-Prime-amino RNA) nucleotide
  modified_base order(1..4,15,16,19,20,22..25,33..36): mod_base = OTHER; note = Phosphorothioate linkages
nnnnnnnnnnnnnnnannnnngttttagagctatgct

SEQ ID NO: 130 - RNA, 38 nt, synthetic construct
  source 1..38: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(16,19): mod_base = OTHER; note = 2-Prime-NH2 (2-Prime-amino RNA) nucleotides
  modified_base order(1..4,15..17,19,20,22..25,33..36): mod_base = OTHER; note = Phosphorothioate linkages
nnnnnnnnnnnnnnnannnanngttttagagctatgct

SEQ ID NO: 131 - RNA, 12 nt, synthetic construct
  source 1..12: mol_type = other RNA
  modified_base 1..12: mod_base = OTHER; note = 2-Prime-O-methyl RNA nucleotides
aaaaccgaggca

SEQ ID NO: 132 - RNA, 13 nt, synthetic construct
  source 1..13: mol_type = other RNA
  modified_base 1..13: mod_base = OTHER; note = 2-Prime-O-methyl RNA nucleotides
taaaaccgaggca

SEQ ID NO: 133 - RNA, 13 nt, synthetic construct
  source 1..13: mol_type = other RNA
  modified_base 1..13: mod_base = OTHER; note = 2-Prime-O-methyl RNA nucleotides
aaaaccgaggcag

SEQ ID NO: 134 - RNA, 14 nt, synthetic construct
  source 1..14: mol_type = other RNA
  modified_base 1..14: mod_base = OTHER; note = 2-Prime-O-methyl RNA nucleotides
taaaaccgaggcag

SEQ ID NO: 135 - RNA, 15 nt, synthetic construct
  source 1..15: mol_type = other RNA
  modified_base 1..15: mod_base = OTHER; note = 2-Prime-O-methyl RNA nucleotides
ctaaaaccgaggcag

SEQ ID NO: 136 - RNA, 15 nt, synthetic construct
  source 1..15: mol_type = other RNA
  modified_base 1..15: mod_base = OTHER; note = 2-Prime-O-methyl RNA nucleotides
taaaaccgaggcagg

SEQ ID NO: 137 - RNA, 16 nt, synthetic construct
  source 1..16: mol_type = other RNA
  modified_base 1..16: mod_base = OTHER; note = 2-Prime-O-methyl RNA nucleotides
ctaaaaccgaggcagg

SEQ ID NO: 138 - DNA, 16 bp, synthetic construct
  source 1..16: mol_type = other DNA
  modified_base 1..14: mod_base = OTHER; note = 2-Prime-O-methyl RNA nucleotides
  modified_base 15..16: mod_base = OTHER; note = dT overhangs
  misc_feature 1..14: note = RNA
  misc_feature 15..16: note = DNA
taaaaccgaggcagtt

SEQ ID NO: 139 - RNA, 14 nt, synthetic construct
  source 1..14: mol_type = other RNA
  modified_base order(1,2,13,14): mod_base = OTHER; note = Locked nucleic acids
  modified_base 3..12: mod_base = OTHER; note = 2-Prime-O-methyl RNA nucleotides
taaaaccgaggcag

SEQ ID NO: 140 - RNA, 14 nt, synthetic construct
  source 1..14: mol_type = other RNA
  modified_base order(1,14): mod_base = OTHER; note = Locked nucleic acids
  modified_base 2..13: mod_base = OTHER; note = 2-Prime-O-methyl RNA nucleotides
taaaaccgaggcag

SEQ ID NO: 141 - RNA, 14 nt, synthetic construct
  source 1..14: mol_type = other RNA
  modified_base 1..14: mod_base = OTHER; note = 2-Prime-O-methyl RNA nucleotides
taaaacaccacatg

SEQ ID NO: 142 - RNA, 15 nt, synthetic construct
  source 1..15: mol_type = other RNA
  modified_base 1..15: mod_base = OTHER; note = 2-Prime-O-methyl RNA nucleotides
ctaaaacaccacatg

SEQ ID NO: 143 - RNA, 15 nt, synthetic construct
  source 1..15: mol_type = other RNA
  modified_base 1..15: mod_base = OTHER; note = 2-Prime-O-methyl RNA nucleotides
taaaacaccacatga

SEQ ID NO: 144 - RNA, 16 nt, synthetic construct
  source 1..16: mol_type = other RNA
  modified_base 1..16: mod_base = OTHER; note = 2-Prime-O-methyl RNA nucleotides
ctaaaacaccacatga

SEQ ID NO: 145 - DNA, 16 bp, synthetic construct
  misc_feature 15..16: note = DNA
  misc_feature 1..14: note = RNA
  source 1..16: mol_type = other DNA
  modified_base 1..14: mod_base = OTHER; note = 2-Prime-O-methyl RNA nucleotides
  modified_base 15..16: mod_base = OTHER; note = dT overhangs
taaaacaccacatgtt

SEQ ID NO: 146 - DNA, 17 bp, synthetic construct
  misc_feature 16..17: note = DNA
  misc_feature 1..15: note = RNA
  source 1..17: mol_type = other DNA
  modified_base 1..15: mod_base = OTHER; note = 2-Prime-O-methyl RNA nucleotides
  modified_base 16..17: mod_base = OTHER; note = dT overhangs
ctaaaacaccacatgtt

SEQ ID NO: 147 - DNA, 17 bp, synthetic construct
  misc_feature 16..17: note = DNA
  misc_feature 1..15: note = RNA
  source 1..17: mol_type = other DNA
  modified_base 1..15: mod_base = OTHER; note = 2-Prime-O-methyl RNA nucleotides
  modified_base 16..17: mod_base = OTHER; note = dT overhangs
taaaacaccacatgatt

SEQ ID NO: 148 - DNA, 18 bp, synthetic construct
  misc_feature 17..18: note = DNA
  misc_feature 1..16: note = RNA
  source 1..18: mol_type = other DNA
  modified_base 1..16: mod_base = OTHER; note = 2-Prime-O-methyl RNA nucleotides
  modified_base 17..18: mod_base = OTHER; note = dT overhangs
ctaaaacaccacatgatt

SEQ ID NO: 149 - RNA, 14 nt, synthetic construct
  source 1..14: mol_type = other RNA
  modified_base 1..14: mod_base = OTHER; note = 2-Prime-O-methyl RNA nucleotides
  modified_base order(1..3,12..14): mod_base = OTHER; note = Phosphorothioate linkages
taaaacaccacatg

SEQ ID NO: 150 - RNA, 14 nt, synthetic construct
  source 1..14: mol_type = other RNA
  modified_base 1..14: mod_base = OTHER; note = 2-Prime-O-methyl RNA nucleotides
  modified_base order(1..4,11..14): mod_base = OTHER; note = Phosphorothioate linkages
taaaacaccacatg

SEQ ID NO: 151 - DNA, 16 bp, synthetic construct
  misc_feature 15..16: note = DNA
  misc_feature 1..14: note = RNA
  source 1..16: mol_type = other DNA
  modified_base 1..14: mod_base = OTHER; note = 2-Prime-O-methyl RNA nucleotides
  modified_base 15..16: mod_base = OTHER; note = dT overhangs
  modified_base order(1..3,12..14): mod_base = OTHER; note = Phosphorothioate linkages
taaaacaccacatgtt

SEQ ID NO: 152 - RNA, 37 nt, synthetic construct
  source 1..37: mol_type = other RNA
  modified_base order(1..3,33..37): mod_base = OTHER; note = 2-Prime-O-methyl RNA nucleotides
  modified_base order(1..4,33,34,35..37): mod_base = OTHER; note = Phosphorothioate linkages
gagacaaatcacctgcctcggttttagagctatgnct

SEQ ID NO: 153 - RNA, 36 nt, synthetic construct; identical to SEQ ID NO: 4
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,20,22..25,33..36): mod_base = OTHER; note = Phosphorothioate linkages

SEQ ID NO: 154 - RNA, 36 nt, synthetic construct
  source 1..36: mol_type = other RNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,20,22..25,33..36): mod_base = OTHER; note = Phosphorothioate linkages
gtcgtgctacttcatgtggtgttttagagctatgct

SEQ ID NO: 155 - DNA, 38 bp, synthetic construct
  misc_feature 1..36: note = RNA
  misc_feature 37..38: note = DNA
  source 1..38: mol_type = other DNA
  modified_base order(1..10,20,21,27..36): mod_base = OTHER; note = 2-Prime-O-methyl RNA nucleotides
  modified_base order(11..14,17,18,25,26): mod_base = OTHER; note = 2-Prime-fluoro RNA nucleotides
  modified_base order(1..4,15..17,19,20,22..25,33..36): mod_base = OTHER; note = Phosphorothioate linkages
  modified_base 37..38: mod_base = OTHER; note = dT overhangs
gtcgtgctacttcatgtggtgttttagagctatgcttt

SEQ ID NO: 156 - RNA, 22 nt, synthetic construct
  source 1..22: mol_type = other RNA
gcacaaaaacnnnnnnnnnnnn

SEQ ID NO: 157 - RNA, 16 nt, synthetic construct
  source 1..16: mol_type = other RNA
accatagctctaaaac

SEQ ID NO: 158 - RNA, 25 nt, synthetic construct
  source 1..25: mol_type = other RNA
cccaaaaaaaatctcccaaagctcc

SEQ ID NO: 159 - RNA, 28 nt, synthetic construct
  source 1..28: mol_type = other RNA
cgggctggaaaaaaaaagctgttcgcgc

SEQ ID NO: 160 - RNA, 25 nt, synthetic construct
  source 1..25: mol_type = other RNA
cccggaaaaaatggcttttggctag

SEQ ID NO: 161 - RNA, 25 nt, synthetic construct
  source 1..25: mol_type = other RNA
cccataaaaaaccttggagcaacgg

SEQ ID NO: 162 - RNA, 10 nt, synthetic construct
  source 1..10: mol_type = other RNA
aaaccgaggc

SEQ ID NO: 163 - RNA, 11 nt, synthetic construct
  source 1..11: mol_type = other RNA
aaaaccgaggc

SEQ ID NO: 164 - RNA, 11 nt, synthetic construct
  source 1..11: mol_type = other RNA
aaaccgaggca

SEQ ID NO: 165 - RNA, 12 nt, synthetic construct
  source 1..12: mol_type = other RNA
aaaaccgaggca

SEQ ID NO: 166 - RNA, 13 nt, synthetic construct
  source 1..13: mol_type = other RNA
taaaaccgaggca

SEQ ID NO: 167 - RNA, 13 nt, synthetic construct
  source 1..13: mol_type = other RNA
aaaaccgaggcag

SEQ ID NO: 168 - RNA, 14 nt, synthetic construct
  source 1..14: mol_type = other RNA
taaaaccgaggcag

SEQ ID NO: 169 - RNA, 15 nt, synthetic construct
  source 1..15: mol_type = other RNA
ctaaaaccgaggcag

SEQ ID NO: 170 - RNA, 15 nt, synthetic construct
  source 1..15: mol_type = other RNA
taaaaccgaggcagg

SEQ ID NO: 171 - RNA, 16 nt, synthetic construct
  source 1..16: mol_type = other RNA
ctaaaaccgaggcagg

SEQ ID NO: 172 - RNA, 23 nt, synthetic construct
  source 1..23: mol_type = other RNA
actggagcagtaatgcctggtgg

SEQ ID NO: 173 - RNA, 28 nt, synthetic construct
  source 1..28: mol_type = other RNA
cccaaaataaaaaaccttggagcaacgg

SEQ ID NO: 174 - RNA, 36 nt, synthetic construct; identical to SEQ ID NO: 4
  source 1..36: mol_type = other RNA
  modified_base order(1..10,20,21,27..36): mod_base = OTHER; note = 2-Prime-O-Me nucleotides
  modified_base order(11..14,17,18,25,26): mod_base = OTHER; note = 2-Prime-F nucleotides
  modified_base order(15,16,19,22..24): mod_base = OTHER; note = 2-Prime-OH nucleotides
  modified_base order(1..3,15,16,19,22..24,33..35): mod_base = OTHER; note = Phosphorothioate linkages

(19) United States
(12) Patent Application Publication
(10) Pub. No.: US 2023/0407295 A1
(43) Pub. Date: Dec. 21, 2023

(54) PROTECTING OLIGONUCLEOTIDES FOR CRISPR GUIDE RNA

(71) Applicant: UNIVERSITY OF MASSACHUSETTS, Boston, MA (US)

(72) Inventors: Han Zhang, Worcester, MA (US); Erik Joseph Sontheimer, Auburndale, MA (US); Jonathan Kenneth Watts, Worcester, MA (US); Anastasia Khvorova, Westborough, MA (US)

(21) Appl. No.: 18/141,166

(22) Filed: Apr. 28, 2023

Related U.S. Application Data
(60) Provisional application No. 63/438,842, filed on Jan. 13, 2023.
(60) Provisional application No. 63/336,340, filed on Apr. 29, 2022.

(51) Int. Cl. C12N 15/11; C12N 9/22; C12N 15/90

(52) U.S. Cl. CPC C12N 15/11; C12N 9/22; C12N 15/907; C12N 2310/322; C12N 2310/315; C12N 2310/321; C12N 2310/20

(57) ABSTRACT

Protecting oligonucleotides are provided. Protecting oligonucleotides with 5′ and/or 3′ conjugated moieties are provided. Protecting oligonucleotides with chemical modifications are provided. Methods of using the protecting oligonucleotides for genome editing with a CRISPR nuclease and kits for performing the same are also provided.

### CROSS REFERENCE TO RELATED APPLICATIONS

This application claims the benefit of U.S. Provisional Patent Application Ser. No. 63/336,340, filed Apr. 29, 2022, and U.S. Provisional Patent Application Ser. No. 63/438,842, filed Jan. 13, 2023. The entire contents of the above-referenced patent applications are incorporated by reference in their entirety herein.

### STATEMENT REGARDING FEDERALLY SPONSORED RESEARCH OR DEVELOPMENT

This invention was made with government support under Grant No. TR002668 awarded by the National Institutes of Health. The Government has certain rights in the invention.

### SEQUENCE LISTING

The instant application contains a Sequence Listing which has been submitted electronically in XML format and is hereby incorporated by reference in its entirety. Said XML file, created on Aug. 25, 2023, is named 740096_UM9-285_ST26.xml and is 438,226 bytes in size.

### FIELD OF THE INVENTION

This disclosure relates to compositions and methods of protecting oligonucleotides for modified guide RNAs for CRISPR genome editing.

### BACKGROUND

CRISPR RNA-guided genome engineering has revolutionized research into human genetic disease and many other aspects of biology. Numerous CRISPR-based in vivo or ex vivo genome editing therapies are nearing clinical trials. At the heart of this revolution are the microbial effector proteins found in class II CRISPR-Cas systems such as Cas9 (type II) and Cas12a/Cpf1 (type V) (Jinek et al. Science 337, 816-821 (2012); Gasiunas et al. PNAS 109, E2579-E2586 (2012); Zetsche et al. Cell 163, 759-771 (2015)).

The most widely used genome editing tool is the type II-A Cas9 from Streptococcus pyogenes strain SF370 (SpCas9) (Jinek et al, supra). Cas9 forms a ribonucleoprotein (RNP) complex with a CRISPR RNA (crRNA) and a trans-activating crRNA (tracrRNA) for efficient DNA cleavage both in bacteria and eukaryotes (FIG. 1). The crRNA contains a guide sequence that directs the Cas9 RNP to a specific locus via base-pairing with the target DNA to form an R-loop. This process requires the prior recognition of a protospacer adjacent motif (PAM), which for SpCas9 is NGG. R-loop formation activates the His-Asn-His (HNH) and RuvC-like endonuclease domains that cleave the target strand and the non-target strand of the DNA, respectively, resulting in a double-strand break (DSB).

For mammalian applications, Cas9 and its guide RNAs can be expressed from DNA (e.g. a viral vector), RNA (e.g. Cas9 mRNA plus guide RNAs in a lipid nanoparticle), or introduced as a ribonucleoprotein (RNP).

Unmodified single-stranded RNAs are extremely vulnerable to nuclease degradation and are rapidly degraded in cells and biological fluids such as serum. Delivering naked and unformulated crRNAs is similar to the scenario of delivering other therapeutic oligonucleotides such as antisense oligonucleotide (ASO) and short interfering RNA (siRNA), where chemical modifications are the key to in vivo efficacy. However, while complete chemical modifications significantly enhanced crRNA stability (FIG. 1C), they compromised the editing efficiency (FIG. 1D). Here we report that short, fully chemically stabilized oligos (hereafter referred to as “protecting oligos”) that are complementary to the under-modified regions of the crRNA significantly enhanced crRNA stability without compromising the activity. Furthermore, we show that the protecting oligos can enhance the potency of crRNAs and tolerate a wide range of conjugations, and may therefore improve delivery and biodistribution. We anticipate that the protecting oligos will enable in vivo delivery of naked crRNA with enhanced stability, activity, and cellular uptake.

### SUMMARY

The present disclosure provides protecting oligonucleotides for CRISPR genome editing. In certain embodiments, the protecting oligonucleotides of the disclosure are heavily or fully chemically modified. The protecting oligonucleotides of the disclosure may confer several advantages in vivo or ex vivo, including crRNA stability, improved potency, and/or reduced off-target effects. Furthermore, in certain embodiments, the protecting oligonucleotides of the disclosure have reduced immunogenicity, e.g., a reduced ability to induce innate immune responses.

In certain aspects, the disclosure provides a protecting oligonucleotide comprising: (a) a sequence that is complementary to a crRNA; and (b) at least one chemically modified nucleotide, wherein the protecting oligonucleotide is capable of binding the crRNA, and wherein the protecting oligonucleotide confers nuclease resistance to the crRNA when bound.

In an embodiment, the modified nucleotides each independently comprise a modification of a ribose group, a phosphate group, a nucleobase, or a combination thereof.

In an embodiment, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In an embodiment, at least 80% of the ribose groups are chemically modified. In an embodiment, at least 90% of the ribose groups are chemically modified. In an embodiment, 100% of the ribose groups are chemically modified.

In an embodiment, crRNA comprises a guide sequence portion capable of hybridizing to a target polynucleotide sequence and a repeat sequence portion.

In an embodiment, the protecting oligonucleotide binds to the crRNA to forma duplex.

In an embodiment, the duplex has a melting temperature (Tm) of greater than 37° C. over the full length of the duplex.

In an embodiment, the duplex has a melting temperature (Tm) of less than 37° C. over the region of complementarity comprising the guide sequence portion.

In an embodiment, the binding of a tracrRNA to the guide sequence portion of the crRNA dissociates the protecting oligonucleotide from the crRNA.

In an embodiment, the protecting oligonucleotide further comprises at least one moiety conjugated to the protecting oligonucleotide. In an embodiment, the at least one moiety is conjugated to at least one of the 5′ end of the protecting oligonucleotide and/or the 3′ end of the protecting oligonucleotide.

In an embodiment, the at least one moiety increases cellular uptake of the guide RNA. In an embodiment, the at least one moiety promotes specific tissue distribution of the guide RNA.

In an embodiment, the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains.

In an embodiment, the at least one moiety is selected from the group consisting of cholesterol, docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3.

In an embodiment, the at least one moiety is conjugated to the guide RNA via a linker. In an embodiment, the linker is selected from the group consisting of an ethylene glycol chain, an alkyl chain, a polypeptide, a polysaccharide, and a block copolymer.

In an embodiment, the at least one moiety is a modified lipid. In an embodiment, the modified lipid is a branched lipid.

In an embodiment, the modified lipid is a branched lipid of Formula I, Formula I: X-MC(═Y)M-Z-[L-MC(═Y)M-R]n, where X is a moiety that links the lipid to the guide RNA, each Y is independently oxygen or sulfur, each M is independently CH₂, NH, O or S, Z is a branching group which allows two or three (“n”) chains to be joined to a chemically modified guide RNA, L is an optional linker moiety, and each R is independently a saturated, monounsaturated or polyunsaturated linear or branched moiety from 2 to 30 atoms in length, a sterol, or other hydrophobic group. In an embodiment, the modified lipid is a headgroup-modified lipid.

In an embodiment, the modified lipid is a headgroup-modified lipid of Formula II, Formula II: X-MC(═Y)M-Z-[L-MC(═Y)M-R]n-L-K-J, where X is a moiety that links the lipid to the guide RNA, each Y is independently oxygen or sulfur, each M is independently CH₂, NH, N-alkyl, O or S, Z is a branching group which allows two or three (“n”) chains to be joined to chemically modified guide RNA, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

In one aspect, the disclosure provides a double stranded oligonucleotide comprising: (a) a crRNA comprising (i) a guide sequence portion capable of hybridizing to a target polynucleotide sequence, and (ii) a repeat sequence portion; and (b) a protecting oligonucleotide that is complementary to the crRNA, wherein the crRNA comprises at least 50% modified nucleotides, and wherein the protecting oligonucleotide comprises at least one modified nucleotide.

In an embodiment, the modified nucleotide comprises a modification of a ribose group, a phosphate group, a nucleobase, or a combination thereof.

In an embodiment, each modification of the ribose group is independently selected from the group consisting of 2′-O-methyl, 2′-fluoro, 2′-deoxy, 2′-O-(2-methoxyethyl) (MOE), 2′-NH₂ (2′-amino), 4′-thio, a bicyclic nucleotide, a locked nucleic acid (LNA), a 2′-(S)-constrained ethyl (S-cEt), a constrained MOE, or a 2′-O,4′-C-aminomethylene bridged nucleic acid (2′,4′-BNA^{NC}).

In an embodiment, at least 80% of the ribose groups are chemically modified. In an embodiment, at least 90% of the ribose groups are chemically modified. In an embodiment, 100% of the ribose groups are chemically modified.

In an embodiment, the double stranded oligonucleotide comprises a melting temperature (Tm) of greater than 37° C. over the full length of the double stranded oligonucleotide.

In an embodiment, the double stranded oligonucleotide comprises a melting temperature (Tm) of less than 37° C. for the region of the double stranded oligonucleotide comprising the crRNA guide sequence portion.

In an embodiment, the binding of a tracrRNA to the repeat sequence portion of the crRNA dissociates the protecting oligonucleotide from the crRNA.

In an embodiment, the protecting oligonucleotide binds to a region of the crRNA that is not fully chemically modified.

In an embodiment, the protecting oligonucleotide binds to a region of the crRNA that comprises less than 100% modified ribose groups.

In an embodiment, the double stranded oligonucleotide further comprises at least one moiety conjugated to the protecting oligonucleotide and/or crRNA. In an embodiment, the at least one moiety is conjugated to at least one of the 5′ end and/or the 3′ end of the protecting oligonucleotide and/or crRNA.

In an embodiment, the at least one moiety increases cellular uptake of the guide RNA. In an embodiment, the at least one moiety promotes specific tissue distribution of the guide RNA.

In an embodiment, the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains.

In an embodiment, the at least one moiety is selected from the group consisting of cholesterol, docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3.

In an embodiment, the at least one moiety is conjugated to the guide RNA via a linker. In an embodiment, the linker is selected from the group consisting of an ethylene glycol chain, an alkyl chain, a polypeptide, a polysaccharide, and a block copolymer.

In an embodiment, the at least one moiety is a modified lipid. In an embodiment, the modified lipid is a branched lipid.

In an embodiment, the modified lipid is a branched lipid of Formula I, Formula I: X-MC(═Y)M-Z-[L-MC(═Y)M-R]n, where X is a moiety that links the lipid to the guide RNA, each Y is independently oxygen or sulfur, each M is independently CH₂, NH, O or S, Z is a branching group which allows two or three (“n”) chains to be joined to a chemically modified guide RNA, L is an optional linker moiety, and each R is independently a saturated, monounsaturated or polyunsaturated linear or branched moiety from 2 to 30 atoms in length, a sterol, or other hydrophobic group. In an embodiment, the modified lipid is a headgroup-modified lipid.

In an embodiment, the modified lipid is a headgroup-modified lipid of Formula II, Formula II: X-MC(═Y)M-Z-[L-MC(═Y)M-R]n-L-K-J, where X is a moiety that links the lipid to the guide RNA, each Y is independently oxygen or sulfur, each M is independently CH₂, NH, N-alkyl, O or S, Z is a branching group which allows two or three (“n”) chains to be joined to chemically modified guide RNA, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

In an embodiment, the guide RNA binds to a Cas9 nuclease selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In an embodiment, the Cas9 is a variant Cas9 with altered activity.

In an embodiment, the variant Cas9 is selected from the group consisting of a Cas9 nickase (nCas9), a catalytically dead Cas9 (dCas9), a hyper accurate Cas9 (HypaCas9), a high fidelity Cas9 (Cas9-HF), an enhanced specificity Cas9 (eCas9), and an expanded PAM Cas9 (xCas9).

In an embodiment, the Cas9 off-target activity is reduced relative to an unmodified guide RNA.

In an embodiment, the Cas9 on-target activity is increased relative to an unmodified guide RNA.

In one aspect, the disclosure provides a method of editing a target region of a genome in a cell altering expression of a target gene in a cell, comprising administering to said cell a genome editing system comprising: a protecting oligonucleotide of any of the embodiments recited above; one or more crRNAs of any of the embodiments recited above; one or more tracrRNAs; and an RNA-guided nuclease or a polynucleotide encoding an RNA-guided nuclease.

In an embodiment, the target gene is in a cell in an organism.

In an embodiment, expression of the target gene is knocked out or knocked down.

In an embodiment, the RNA-guided nuclease or a polynucleotide encoding an RNA-guided nuclease and the one or more tracrRNAs are administered to the cell before the one or more crRNAs or the protecting oligonucleotide.

In an embodiment, the crRNA comprises a crRNA portion of any of the preceding embodiments modification pattern consisting of:

(SEQ ID NO: 3)
mN#mN#mN#mNmNmNmNmNmNmNfNfNfNfNrN#rN#fNfNr
N#mNmGrU#rU#rU#fUfAmGmAmGmCmUmAmU#mG#mC#mU
(crRNA 20),

wherein rN=RNA, mN=2′-O-methyl RNA, fN=2′-fluoro RNA, N # N=phosphorothioate linkage, and N=any nucleotide.

In an embodiment, the protecting oligonucleotide of any of the preceding embodiments comprises a modification pattern consisting of:

mAmAmAmCmNmNmNmNmN (RC01);
mAmAmAmAmCmNmNmNmNmN (RC02);
mAmAmAmCmNmNmNmNmNmN (RC03);
mAmAmAmAmCmNmNmNmNmNmN (RC04);
mUmAmAmAmAmCmNmNmNmNmNmN (RC05);
mAmAmAmAmCmNmNmNmNmNmNmN (RC06);
mUmAmAmAmAmCmNmNmNmNmNmNmN (RC07);
mCmUmAmAmAmAmCmNmNmNmNmNmNmN (RC08);
mUmAmAmAmAmCmNmNmNmNmNmNmNmN (RC09);
mCmUmAmAmAmAmCmNmNmNmNmNmNmNmN (RC10);
mU#mA#mAmAmAmCmNmNmNmNmN#mN#mN (RC07-2PS);
and
mU#mA#mA#mAmAmCmNmNmNmN#mN#mN#mN (RC07-3PS),

wherein rN=RNA, mN=2′-O-methyl RNA, N # N=phosphorothioate linkage, and N=any nucleotide.

In certain embodiments, the protecting oligonucleotide of any one of the preceding embodiments is a protecting oligonucleotide modification pattern selected from any of RC01-10 of Table 1.

In certain embodiments, the crRNA of any one of the preceding embodiments is a crRNA modification pattern selected from any of crRNA 1-134 of Table 2.

In certain embodiments, protecting oligonucleotide and/or crRNA of any one of the preceding embodiments further comprises one or more additional modified nucleotides, each independently selected from a modification of a ribose group, a phosphate group, a nucleobase, or a combination thereof.

In certain embodiments, each modification of the ribose group is independently selected from the group consisting of 2′-O-methyl, 2′-fluoro, 2′-deoxy, 2′-O-(2-methoxyethyl) (MOE), 4′-thio, a bicyclic nucleotide, a locked nucleic acid (LNA), a 2′-(S)-constrained ethyl (S-cEt), a constrained MOE, and a 2′-O,4′-C-aminomethylene bridged nucleic acid (2′,4′-BNA^{NC}).

In certain embodiments, each modification of the phosphate group is independently selected from the group consisting of a phosphorothioate, phosphonoacetate (PACE), thiophosphonoacetate (thioPACE), amide, triazole, phosphonate, or phosphotriester modification.

In certain embodiments, each modification of the nucleobase group is independently selected from the group consisting of 2-thiouridine, 4-thiouridine, N⁶ -methyladenosine, pseudouridine, 2,6-diaminopurine, inosine, thymidine, 5-methylcytosine, 5-substituted pyrimidine, isoguanine, isocytosine, and halogenated aromatic groups.

### BRIEF DESCRIPTION OF THE DRAWINGS

The foregoing and other features and advantages of the present disclosure will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1A-FIG. 1E Schematic representation of stable cell lines, and activity and stability of chemically modified crRNAs. FIG. 1A is a schematic representation of the stable HEK293T cell line that expresses SpyCas9 nuclease, tracrRNA, and a fluorescent reporter gene that consists of a broken GFP and an out-of-frame mCherry. Electroporation of crRNAs targeting the GFP sequence will generate frameshift mutations and place the mCherry coding sequence in-frame. FIG. 1B is a schematic representation of the stable HEK293T cell line that expresses SpyCas9 Adenine base editor (SpyCas9-ABE8e), tracrRNA, and a fluorescent reporter gene that consists of a G-to-A nonsense mutation within the GFP coding sequence. Electroporation of crRNA will restore GFP fluorescence by an A-to-G edit that reverses the mutation. FIG. 1C depicts the activities of different chemically modified crRNAs were measured by electroporating 50 pmole crRNA per 5×10⁴ cells to the stable cell line shown in FIG. 1A. Cells were harvested 48 hours post-electroporation and the percentage of mCherry positive cells was quantified by FACS. The crRNA sequence employed is GAGACAAAUCACCUGCCUCGGUUUUAGAGCUAUGCU (SEQ ID NO:4) and the chemical modification patterns of the crRNAs are recited in Table 2. FIG. 1D (SEQ ID NOS:158-161) depict activities of different chemically modified crRNAs measured by electroporating 50 pmole crRNA per 5×10⁴ cells in a stable mouse Hepal -6 cell line that expresses SpyCas9-ABE8e and tracrRNA. Data measured by Sanger sequencing and EditR analysis. FIG. 1E depicts the stability of partially and fully-modified crRNAs shown in FIG. 1C. crRNAs were diluted to 1 μM concentration in 10% FBS and incubated at 37° C. for the indicated numbers of hours before denaturing by formamide loading dye and fractionation by 10% urea-denaturing polyacrylamide gel electrophoresis (PAGE) followed by staining.

FIG. 2A-FIG. 2C depict protecting oligos enhancing the stability of crRNA without compromising activity. FIG. 2A (SEQ ID NOS:162-171,174) depicts representative designs and chemical modifications of C20 (top) and protecting oligos (bottom, RC01 to RC10). Chemical modification patterns of C20 and protecting oligos are color-coded: green, 2′-O-Me; red 2′-F; black, 2′-OH; underlined, PS. FIG. 2B depicts the stability of C20-protecting oligo complex. C20 and the indicated protecting oligo were mixed at equal molar ratio and annealed by heating to 95° C. for 5 minutes and slowly cooled down to 4° C. (-0.1° C. per second). Annealed C20-protecting oligo complex was diluted to 1 μM in 10% FBS and incubated for the indicated number of hours at 37° C. The RNAs were then denatured by formamide and resolved by 10% denaturing urea PAGE. FIG. 2C depicts the activity of the C20-protecting oligo complex. C20 and the indicated protecting oligo were annealed as described in FIG. 2B. 50 pmole crRNA per 5×10⁴ cells was then electroporated using the stable cell line shown in FIG. 1A. Cells were harvested at 48 hours post-electroporation and the percentage of mCherry was quantified by FACS.

FIG. 3A-FIG. 3B depicts protecting oligos enhancing the potency of crRNA. FIG. 3A depicts the activity of the C20-protecting oligo complex in the stable cell line shown in FIG. 1A at non-saturating dosages. FIG. 3B depicts the activity of C20-protecting oligo complex in the stable cell line shown in FIG. 1B at non-saturating dosages. The molar amounts of C20-protecting oligo complex per 5×10⁴ cells are given on the x axis. Cells were harvested at 48 hours post-electroporation and the percentage of mCherry or GFP positive cells was quantified by FACS.

FIG. 4A-FIG. 4D depicts the conjugation of protecting oligos enhanced cellular uptake without interfering with crRNA activity. FIG. 4A depicts the activities of C20-protecting oligo complex with conjugates were measured in the stable cell line shown in FIG. 1B by electroporating 50 pmole complex per 5×10⁴ cells. The chemical modification pattern of C20 (“crRNA 20”) is recited in Table 2 and the chemical modification pattern of the protecting oligo RC10 is recited in Table 1. Cells were harvested at 48 hours post-electroporation and the percentage of mCherry or GFP positive cells was quantified by FACS. FIG. 4B depicts a schematic representation of the design and modifications the editing efficiency of protecting oligonucleotides with and without lipid conjugations (Cholesterol is Chol, docosahexaenoic acid is DHA, N-acetylgalactosamine is GalNac) by GFP quantification. FIG. 4C depicts C20-protecting oligo complex uptake in mouse CNS after intrastriatal injection (n =3 mice per group).

FIG. 4D depicts cellular uptake of Cy3-labeled crRNA (right), Cy3-labeled crRNA with protecting oligo (middle), and with Cholesterol-conjugated protecting oligo (left). Oligonucleotides were diluted to 1.5 μM in DMEM+3% FBS then mixed with HEK293T cells. Cells were plated on an 8-well glass bottom cell culture chamber slide (SKU230118) and incubated overnight. Cell culture media was then replaced with PBS and one drop of NucBlue (Hoechst 33342) were added to cell culture before imaging. Images were taken under Leica fluorescent microscope at 40× magnification.

FIG. 5 depicts editing efficiency by passive uptake of C20 with and without protecting oligo. GFP positive cells were quantified by FACS.

FIG. 6 depicts optimal duplex stability between protecting oligo and crRNA may be required for efficient editing. The activities of C20-protecting, oligo complexes in the stable cell line shown in FIG. 1B. C20 and the indicated protecting oligo were annealed as described in FIG. 1A. 50 pmole crRNA per 5×10⁴ cells was then electroporated using the stable cell line shown in FIG. 1A. Cells were harvested at 48 hours post-electroporation and the percentage of mCherry was quantified by FACS. The chemical modification patterns of C20 and the protecting oligos were color-coded: green, 2′-O-Me; red, 2′-F; black: 2′-OH; underlined: PS, double underlined: locked nucleic acid (LNA).

FIG. 7A-FIG. 7B depict the in vivo genome editing in adult mouse CNS by co-delivery of AAV and self-delivering crRNA. FIG. 7A depicts the process of genome editing in adult mouse using scAAV9 by injection up to immunohistochemistry (IHC) detection. The editing efficiencies of these conjugates were tested by IHC staining using an anti-EGFP antibody and were quantified by percentage of EGFP positive cells within 1 mm2 around the site-of-injection. These results are shown in FIG. 7B. The chemical modification patterns are recited in Table 1.

FIG. 8A-FIG. 8G depict the in vivo genome editing in adult mouse liver by co-delivery of AAV-expressed effector and tracrRNA and GalNac conjugated self-delivering crRNA. The results of the expression of the injected AAV are shown in FIG. 8A for SpyCas9-ABE8e and tracrRNA expression and FIG. 8B for SpyCas9 nuclease and tracrRNA expression. FIG. 8C depicts the Fah^{PM/PM} generating a point mutation resulting in exon 8 skipping and FAH deficiency. FIG. 8D shows the schematic representation of the AAV delivery to the Fah^{PM/PM} mouse. First, AAV were RO injected to express SpyCas9-ABE8e and tracrRNA. After five weeks, RO injections of C20 were performed with a previously validated spacer sequence FIG. 8E depicts base editing efficiency of the genomic DNA of the mouse liver treated by AAV-SpyCas9-ABE-tracrRNA co-delivery with indicated C20/protecting oligo compound. The editing efficiency was measured by targeted amplicon deep sequencing (n=3 mice per group). Data present mean±SD, *, P<0.05; **, P<0.01; ***, P<0.001 (two-way ANOVA). The body weight over time of the mice treated with AAV-expressed SpyCas9-ABE8e and tracrRNA, and C20 with protecting oligonucleotides was measured and the results are shown in FIG. 8F. The mice were then sacrificed after three months of NTBC cycles, and IHC staining was performed on liver sections using anti-FAH antibodies, the staining results are shown in FIG. 8G.

FIG. 9A-FIG. 9B depict the effect of optimization of the dosing regimen on the in vivo editing efficiency. FIG. 9A shows the schematic representation of the experimental process to test repeat crRNA dosing regimen. RO, retro-orbital injection. FIG. 9B depicts the editing efficiency of AAV-SpyCas9-ABE-tracrRNA co-delivered with either a single 80 mg/kg dose, as compared to the editing efficiency of three consecutive daily injections 26 mg/kg dose of C20+RC07-GalNac targeting the mouse Pcsk9 gene. The editing efficiency was measured by targeted amplicon deep sequencing of the mouse liver genomic DNA (n=3 mice per group). The single dose data was the same as what shown in FIG. 8A. Data present mean±SD, ****, P<(two-way ANOVA).

FIG. 10A-FIG. 10B depict the effect of C20 targeting different sequences with and without protecting oligonucleotides. FIG. 10A shows the dose-response curves of C20 targeting different sequences with and without protecting oligonucleotides in the HEK293T-SpyCas9-ABE-tracrRNA-dGFP reporter. The editing efficiency was quantified by FACS. Data represent mean±SD; ns, P>0.05, ****, P<0.0001 (two-way ANOVA, C20 was used as control in multiple comparisons). FIG. 10B Editing efficiency of the fully modified crRNA C40, with and without a 14-mer protecting oligo at different dosages in the SpyCas9-ABE-tracrRNA-dGFP reporter. The left panel shows the chemical modification pattern of the crRNA and the protecting oligo. Data represent mean±SD; ns, P>0.05 (two-way ANOVA).

### DETAILED DESCRIPTION

Provided herewith are protecting oligonucleotides, including heavily or fully chemically protecting oligonucleotides. In certain embodiments, protecting oligonucleotides and crRNAs with 5′ and/or 3′ conjugated moieties are provided. Methods of using the protecting oligonucleotides and crRNAs of the disclosure for genome editing with a CRISPR nuclease and kits for performing the same are also provided.

Unless otherwise defined herein, nomenclature used in connection with cell and tissue culture, molecular biology, immunology, microbiology, genetics and protein and nucleic acid chemistry and hybridization described herein are those well-known and commonly used in the art. The methods and techniques provided herein are usually performed according to conventional methods well known in the art and as described in various general and more specific references that are cited and discussed throughout the present specification unless otherwise indicated. Enzymatic reactions and purification techniques are performed according to manufacturer's specifications unless otherwise specified, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art, unless otherwise specified. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

As used herein, the term “protecting oligonucleotide” refers to an oligonucleotide that comprises complementarity to a crRNA and confers increased nuclease resistance to the crRNA when bound relative to a crRNA that is not bound by a protecting oligonucleotide.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” is the DNA sequence in a genome of a cell that is complementary to the guide sequence of the gRNA.

In addition to the targeting domains, gRNAs typically include a plurality of domains that influence the formation or activity of gRNA/Cas9 complexes. For example, as mentioned above, the duplexed structure formed by first and secondary complementarity domains of a gRNA (also referred to as a repeat: anti-repeat duplex) interacts with the recognition (REC) lobe of Cas9 and may mediate the formation of Cas9/gRNA complexes (Nishimasu et al. Cell 156: 935-949 (2014); Nishimasu et al. Cell 162(2), 1113-1126 (2015), both incorporated by reference herein). It should be noted that the first and/or second complementarity domains can contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for example through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are necessary for nuclease activity in vivo but not necessarily in vitro (Nishimasu 2015, supra). A first stem-loop near the 3′ portion of the second complementarity domain is referred to variously as the “proximal domain,” “stem loop 1” (Nishimasu 2014, supra; Nishimasu 2015, supra) and the “nexus” (Briner 2014, supra). One or more additional stem loop structures are generally present near the 3′ end of the gRNA, with the number varying by species: S. pyogenes gRNAs typically include two 3′ stem loops (for a total of four stem loop structures including the repeat: anti-repeat duplex), while S. aureus and other species have only one (for a total of three). A description of conserved stem loop structures (and gRNA structures more generally) organized by species is provided in Briner 2014, which is incorporated herein by reference. Additional details regarding guide RNAs generally may be found in WO2018026976A1, which is incorporated herein by reference.

### Protecting Oligonucleotides

The inventors have surprisingly discovered that the use of protecting oligonucleotides confers increased nuclease resistance to crRNAs. The protecting oligonucleotides described herein comprise complementarity to a crRNA. The protecting oligonucleotides confer nuclease resistance to the crRNAs when bound. In preferred embodiments, the protecting oligonucleotides described herein bind to a region of the crRNA the contains at least one unmodified ribose group. In certain embodiments, the protecting oligonucleotide binds to a region of the crRNA the contains 1, 2, 3, 4, 5, 6, 7, 8, 9, or 10 unmodified ribose groups.

In certain embodiments, the protecting oligonucleotide is about 5 to about 20 nucleotides in length (e.g., 5, 6, 7, 8, 9, 10, 11, 12, 13, 14, 15, 16, 17, 18, 19, or 20 nucleotides in length). In certain embodiments, the protecting oligonucleotide is 11, 12, 13, 14, 15, or 16 nucleotides in length. The length of the protecting oligonucleotide is shorter than the crRNA to which the protecting oligonucleotide comprises complementarity to (i.e., the protecting oligonucleotide comprises complementarity to a region of the crRNA, wherein said region is not the full length of the crRNA).

In certain embodiments, the protecting oligonucleotide binds to the crRNA to form a duplex, optionally wherein the protecting oligonucleotide binds to a region of the crRNA that is not fully chemically modified.

In certain embodiments, the duplex has a melting temperature (Tm) of greater than 37° C. over the full length of the duplex.

In certain embodiments, the duplex has a melting temperature (Tm) of less than 37° C. over the region of complementarity comprising the guide sequence portion.

In certain embodiments, the binding of a tracrRNA to the guide sequence portion of the crRNA dissociates the protecting oligonucleotide from the crRNA.

Exemplary protecting oligonucleotide sequences are recited below, wherein “N” corresponds to any nucleotide:

AAACNNNNN
AAAACNNNNN
AAACNNNNNN
AAAACNNNNNN
UAAAACNNNNNN
AAAACNNNNNNN
UAAAACNNNNNNN
CUAAAACNNNNNNN
UAAAACNNNNNNNN
CUAAAACNNNNNNNN

### Chemically Modified Guide RNA and Protecting Oligonucleotides

The protecting oligonucleotides and guide RNAs (i.e., crRNAs and tracrRNAs) of the disclosure possess improved in vivo stability, improved genome editing efficacy, and/or reduced immunotoxicity relative to unmodified or minimally modified protecting oligonucleotides and guide RNAs.

The protecting oligonucleotides and guide RNAs of the disclosure contain one or more modified nucleotides comprising a modification in a ribose group, a phosphate group, a nucleobase, or a combination thereof.

Chemical modifications to the ribose group may include, but are not limited to, 2′-O-methyl, 2′-fluoro, 2′-deoxy, 2′-O-(2-methoxy ethyl) (MOE), 2′-NH₂(2′-amino), 4′-thio, 2′-O-Allyl, 2′-O-Ethylamine, 2′-O-Cyanoethyl, 2′-O-Acetalester, or a bicyclic nucleotide, such as locked nucleic acid (LNA), 2′-(S)-constrained ethyl (S-cEt), constrained MOE, or 2′-O,4′-C-aminomethylene bridged nucleic acid (2′,4′-BNA^{NC}).

The term “4′-thio” as used herein corresponds to a ribose group modification where the sugar ring oxygen of the ribose is replaced with a sulfur.

Chemical modifications to the phosphate group may include, but are not limited to, a phosphorothioate, phosphonoacetate (PACE), thiophosphonoacetate (thioPACE), amide, triazole, phosphonate, or phosphotriester modification.

In an embodiment, the crRNA portion of the chemically modified guide RNA comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications). In an embodiment, the crRNA portion of the chemically modified guide RNA comprises between 1 and 20 phosphorothioate modifications (i.e., 1, 2, 3, 4, 5, 6, 7, 8, 9, 10, 11, 12, 13, 14, 15, 16, 17, 18, 19, or 20 phosphorothioate modifications) and comprises at least about 50% activity relative to a guide RNA that does not comprise phosphorothioate modifications (e.g., 50% activity, 60% activity, 70% activity, 80% activity, 90% activity, 95% activity, or 100% activity, relative to a guide RNA that does not comprise phosphorothioate modifications).

Chemical modifications to the nucleobase may include, but are not limited to, 2-thiouridine, 4-thiouridine, N⁶-methyladenosine, pseudouridine, 2,6-diaminopurine, inosine, thymidine, 5-methylcytosine, 5-substituted pyrimidine, isoguanine, isocytosine, or halogenated aromatic groups.

The chemically modified guide RNAs may have one or more chemical modifications in the crRNA portion and/or the tracrRNA portion for a modular or dual RNA guide. The chemically modified guide RNAs may also have one or more chemical modifications in the single guide RNA for the unimolecular guide RNA.

The protecting oligonucleotides and guide RNAs may comprise at least about 50% to at least about 100% chemically modified nucleotides, at least about 60% to at least about 100% chemically modified nucleotides, at least about 70% to at least about 100% chemically modified nucleotides, at least about 80% to at least about 100% chemically modified nucleotides, at least about 90% to at least about 100% chemically modified nucleotides, and at least about 95% to at least about 100% chemically modified nucleotides.

The protecting oligonucleotides and guide RNAs may comprise at least about 50% chemically modified nucleotides, at least about 60% chemically modified nucleotides, at least about 70% chemically modified nucleotides, at least about 80% chemically modified nucleotides, at least about 90% chemically modified nucleotides, at least about 95% chemically modified nucleotides, at least about 99% chemically modified, or 100% (fully) chemically modified nucleotides.

The protecting oligonucleotides and guide RNAs may comprise at least about 80%, 81%, 82%, 83%, 84%, 85%, 86%, 87%, 88%, 89%, 90%, 91%, 92%, 93%, 94%, 95%, 96%, 97%, 98%, 99%, or 100% chemically modified nucleotides.

Protecting oligonucleotides and guide RNAs that comprise at least about 80% chemically modified nucleotides to at least about 99% chemically modified nucleotides are considered “heavily” modified, as used herein.

Protecting oligonucleotides and guide RNAs that comprise 100% chemically modified nucleotides are considered “fully” modified, as used herein.

In certain exemplary embodiments, the protecting oligonucleotides and guide RNAs may comprise a chemically modified ribose group at about 50% of the nucleotides to about 100% of the nucleotides, at about 60% of the nucleotides to about 100% of the nucleotides, at about 70% of the nucleotides to about 100% of the nucleotides, at about 80% of the nucleotides to about 100% of the nucleotides, at about 90% of the nucleotides to about 100% of the nucleotides, and at about 95% of the nucleotides to about 100% of the nucleotides

In certain exemplary embodiments, the protecting oligonucleotides and guide RNAs may comprise a chemically modified ribose group at about 50% of the nucleotides, at about 60% of the nucleotides, at about 70% of the nucleotides, at about 80% of the nucleotides, at about 90% of the nucleotides, at about 95% of the nucleotides, at about 99% of the nucleotides, or at 100% of the nucleotides.

In certain exemplary embodiments, the protecting oligonucleotides and guide RNAs may comprise a chemically modified ribose group at about 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotides.

Protecting oligonucleotides and guide RNAs that have at least about 80% of the ribose groups chemically modified to at least about 99% of the ribose groups chemically modified are considered “heavily” modified, as used herein.

Protecting oligonucleotides and guide RNAs that have 100% of the ribose groups chemically modified are considered “fully” modified, as used herein.

In certain exemplary embodiments, the protecting oligonucleotides and guide RNAs may comprise a chemically modified phosphate group at about 50% of the nucleotides to about 100% of the nucleotides, at about 60% of the nucleotides to about 100% of the nucleotides, at about 70% of the nucleotides to about 100% of the nucleotides, at about 80% of the nucleotides to about 100% of the nucleotides, at about 90% of the nucleotides to about 100% of the nucleotides, and at about 95% of the nucleotides to about 100% of the nucleotides

In certain exemplary embodiments, the protecting oligonucleotides and guide RNAs may comprise a chemically modified phosphate group at about 50% of the nucleotides, at about 60% of the nucleotides, at about 70% of the nucleotides, at about 80% of the nucleotides, at about 90% of the nucleotides, at about 95% of the nucleotides, at about 99% of the nucleotides, or at 100% of the nucleotides.

In certain exemplary embodiments, the protecting oligonucleotides and guide RNAs may comprise a chemically modified phosphate group at about 80%, 81%, 82%, 83%, 84%, 85%, 86%, 87%, 88%, 89%, 90%, 91%, 92%, 93%, 94%, 95%, 96%, 97%, 98%, 99%, or 100% of nucleotides.

Protecting oligonucleotides and guide RNAs that have at least about 80% of the phosphate groups chemically modified to at least about 99% of the phosphate groups chemically modified are considered “heavily” modified, as used herein.

Protecting oligonucleotides and guide RNAs that have 100% of the phosphate groups chemically modified are considered “fully” modified, as used herein.

In certain exemplary embodiments, the protecting oligonucleotides and guide RNAs may comprise a chemically modified nucleobase at about 50% of the nucleotides to about 100% of the nucleotides, at about 60% of the nucleotides to about 100% of the nucleotides, at about 70% of the nucleotides to about 100% of the nucleotides, at about 80% of the nucleotides to about 100% of the nucleotides, at about 90% of the nucleotides to about 100% of the nucleotides, and at about 95% of the nucleotides to about 100% of the nucleotides.

In certain exemplary embodiments, the protecting oligonucleotides and guide RNAs may comprise a chemically modified nucleobase at about 50% of the nucleotides, at about 60% of the nucleotides, at about 70% of the nucleotides, at about 80% of the nucleotides, at about 90% of the nucleotides, at about 95% of the nucleotides, at about 99% of the nucleotides, or at 100% of the nucleotides.

In certain exemplary embodiments, the protecting oligonucleotides and guide RNAs may comprise a chemically modified nucleobase at about 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotides.

Protecting oligonucleotides and guide RNAs that have at least about 80% of the nucleobases chemically modified to at least about 99% of the nucleobases chemically modified are considered “heavily” modified, as used herein.

Protecting oligonucleotides and guide RNAs that have 100% of the nucleobases chemically modified are considered “fully” modified, as used herein.

In certain exemplary embodiments, the protecting oligonucleotides and guide RNAs may comprise any combination of chemically modified ribose groups, chemically modified phosphate groups, and chemically modified nucleobases at about 50% of the nucleotides to about 100% of the nucleotides, at about 60% of the nucleotides to about 100% of the nucleotides, at about 70% of the nucleotides to about 100% of the nucleotides, at about 80% of the nucleotides to about 100% of the nucleotides, at about 90% of the nucleotides to about 100% of the nucleotides, and at about 95% of the nucleotides to about 100% of the nucleotides.

In certain exemplary embodiments, the protecting oligonucleotides and guide RNAs may comprise any combination of chemically modified ribose groups, chemically modified phosphate groups, and chemically modified nucleobases at about 50% of the nucleotides, at about 60% of the nucleotides, at about 70% of the nucleotides, at about 80% of the nucleotides, at about 90% of the nucleotides, at about 95% of the nucleotides, at about 99% of the nucleotides, or at 100% of the nucleotides.

In certain exemplary embodiments, the protecting oligonucleotides and guide RNAs may comprise any combination of chemically modified ribose groups, chemically modified phosphate groups, and chemically modified nucleobases at about 80%, 81%, 82%, 83%, 84%, 85%, 86%, 87%, 88%, 89%, 90%, 91%, 92%, 93%, 94%, 95%, 96%, 97%, 98%, 99%, or 100% of the nucleotides.

Protecting oligonucleotides and guide RNAs that have at least about 80% of any combination of the ribose groups, the phosphate groups, and the nucleobases chemically modified to at least about 99% of the nucleobases chemically modified are considered “heavily” modified, as used herein.

Protecting oligonucleotides and guide RNAs that have 100% of any combination of the ribose groups, the phosphate groups, and the nucleobases chemically modified are considered “fully” modified, as used herein.

The heavily and fully chemically modified protecting oligonucleotides and guide RNAs of the disclosure possess several advantages over the minimally modified protecting oligonucleotides and guide RNAs in the art. Heavily and fully chemically modified protecting oligonucleotides and guide RNAs are expected to ease chemical synthesis, further enhance in vivo stability, and provide a scaffold for terminally appended chemical functionalities that facilitate delivery and efficacy during clinical applications to genome editing.

The chemical modification pattern used in the guide RNA is such that activity of the guide RNA is maintained when paired with an RNA-guided DNA endonuclease, e.g., Cas9.

In an embodiment, the chemically modified guide RNAs of the disclosure comprise at least about 50% activity relative to an unmodified guide RNA (e.g., 50% activity, 60% activity, 70% activity, 80% activity, 90% activity, 95% activity, or 100% activity, relative to an unmodified guide RNA).

The activity of a guide RNA can be readily determined by any means known in the art. In an embodiment, % activity is measured with the traffic light reporter (TLR) Multi-Cas Variant 1 system (TLR-MCV1), described below. The TLR-MCV1 system will provide a % fluorescent cells which is a measure of % activity.

Exemplary chemical modification patterns are described in Table 1 and Table 2 below.

TABLE 1
Exemplary chemical modification patterns
for protecting oligonucleotides.

| Name | Sequence |
|---|---|
| RC01 | mAmAmAmCmNmNmNmNmN |
| RC02 | mAmAmAmAmCmNmNmNmNmN |
| RC03 | mAmAmAmCmNmNmNmNmNmN |
| RC04 | mAmAmAmAmCmNmNmNmNmNmN |
| RC05 | mUmAmAmAmAmCmNmNmNmNmNmN |
| RC06 | mAmAmAmAmCmNmNmNmNmNmNmN |
| RC07 | mUmAmAmAmAmCmNmNmNmNmNmNmN |
| RC08 | mCmUmAmAmAmAmCmNmNmNmNmNmNmN |
| RC09 | mUmAmAmAmAmCmNmNmNmNmNmNmNmN |
| RC10 | mCmUmAmAmAmAmCmNmNmNmNmNmNmNmN |
| RC07-2PS | mU#mA#mAmAmAmCmNmNmNmNmN#mN#mN |
| RC07-3PS | mU#mA#mA#mAmAmCmNmNmNmN#mN#mN#mN |
| RC07-4xLNA | lUlAmAmAmAmCmNmNmNmNmNmNlNlN |
| RC07-2xLNA | lUmAmAmAmAmCmNmNmNmNmNmNmNlN |
| RC07-d(TT)- | mUmAmAmAmAmCmNmNmNmNmNmNmN |
| DHA | dTdT-DHA |
| RC07-d(TT)- | mUmAmAmAmAmCmNmNmNmNmNmNmNdTdT- |
| TegChol | TegChol |

KEY:
rN = RNA,
dN = DNA,
mN = 2′-O-methyl RNA,
N = A, U, G, or C
N#N = phosphorothioate linkage,
l = an LNA modification

TABLE 2
Exemplary chemical modification patterns for crRNAs

| Name | Sequence |
|---|---|
| crRNA 1 | mN#mN#mN#mNmNmNmNmNmNmNrNrNrNrNrNmNmN |
| | mNrNmNmGrUrUrUmUmAmGmAmGmCmUmAmU#mG# |
| | mCmU(SEQ ID NO: 5) |
| crRNA 2 | rNrNrNrNrNrNmNmNmNmNrNrNrNrNrNrNrNrNrNmNm |
| | GrUrUrUrUrAmGmAmGmCmUmAmU#mG#mCmU |
| | (SEQ ID NO: 6) |
| crRNA 3 | rN#rN#rN#rNrNrNmNmNmNmNrNrNrNrNrNrNrNrNr |
| | NmNmGrUrUrUrUrAmGmAmGmCmUmAmU#mG#mCmU |
| | (SEQ ID NO: 7) |
| crRNA 4 | mN#mN#mN#mNmNmNmNmNmNmNrNrNrNrNrNrNrNr |
| | NrNmNmGrUrUrUrUrAmGmAmGmCmUmAmU#mG#mC |
| | #mU(SEQ ID NO: 8) |
| crRNA 5 | rN#rN#rN#rNrNrNmNmNmNmNrNrNrNrNrNrNmNm |
| | NrNmNmGrUrUrUrUrAmGmAmGmCmUmAmU#mG#mC#mU |
| | (SEQ ID NO: 9) |
| crRNA 6 | rN#rN#rN#rNrNrNmNmNmNmNrNrNrNrNrNrNrNr |
| | NrNmNmGrUrUrUmUmAmGmAmGmCmUmAmU#mG#mC#mU |
| | (SEQ ID NO: 10) |
| crRNA 7 | mN#mN#mN#mNmNmNmNmNmNmNrNrNrNrNrNrNmN |
| | mNrNmNmGrUrUrUrUrAmGmAmGmCmUmAmU#mG#m |
| | C#mU(SEQ ID NO: 11) |
| crRNA 8 | mN#mN#mN#mNmNmNmNmNmNmNrNrNrNrNrNrNrNr |
| | NrNmNmGrUrUrUmUmAmGmAmGmCmUmAmU#mG#m |
| | C#mU(SEQ ID NO: 12) |
| crRNA 9 | mN#mN#mN#mNmNmNmNmNmNmNrNrNrNrNrN#rN#r |
| | NrNrN#mNmGrU#rU#rU#mUmAmGmAmGmCmUmAmU |
| | #mG#mC#mU(SEQ ID NO: 13) |
| crRNA 10 | mN#mN#mN#mNmNmNmNmNmNmNfNfNfNfNrN#rN#f |
| | NfNrN#mNmGrU#rU#rU#mUmAmGmAmGmCmUmAmU |
| | #mG#mC#mU(SEQ ID NO: 14) |
| crRNA 11 | mN#mN#mN#mNmNmNmNmNmNmNfNfNfNfNrN#rN#r |
| | N#rN#rN#mNmGrU#rU#rU#mUmAmGmAmGmCmUmAm |
| | U#mG#mC#mU(SEQ ID NO: 15) |
| crRNA 17 | mN#mN#mN#mNmNmNmNmNmNmNfNfNfNfNrN#rN#f |
| | NfNrN#mNmGrU#rU#rU#mUrA#mGmAmGmCmUmAmU |
| | #mG#mC#mU(SEQ ID NO: 16) |
| crRNA 18 | mN#mN#mN#mNmNmNmNmNmNmNfNfNfNfNrN#rN#f |
| | NfNrN#mNmGrU#rU#rU#rU#mAmGmAmGmCmUmAmU |
| | #mG#mC#mU(SEQ ID NO: 17) |
| crRNA 19 | mN#mN#mN#mNmNmNmNmNmNmNfNfNfNfNrN#rN#f |
| | NfNrN#mNmGrU#rU#rU#rU#rA#mGmAmGmCmUmAmU |
| | #mG#mC#mU(SEQ ID NO: 18) |
| crRNA 20 | mN#mN#mN#mNmNmNmNmNmNmNfNfNfNfNrN#rN#f |
| | NfNrN#mNmGrU#rU#rU#fUfAmGmAmGmCmUmAmU# |
| | mG#mC#mU(SEQ ID NO: 3) |
| crRNA 21 | mN#mN#mN#mNmNmNmNmNmNmNfNfNfNfNfNfNfNf |
| | NfNmNmGfUfUfUfUfAmGmAmGmCmUmAmU#mG#mC |
| | #mU(SEQ ID NO: 19) |
| crRNA 22 | mN#mN#mN#mNmNmNmNmNmNmNfNfNfNfNrN#rN#f |
| | NfNrN#mNmGfUrU#fUfUfAmGmAmGmCmUmAmU#mG |
| | #mC#mU(SEQ ID NO: 20) |
| crRNA 23 | mN#mN#mN#mNmNmNmNmNmNmNfNfNfNfNmNrN#f |
| | NfNrN#mNmGrU#rU#rU#fUfAmGmAmGmCmUmAmU# |
| | mG#mC#mU(SEQ ID NO: 21) |
| crRNA 24 | mN#mN#mN#mNmNmNmNmNmNmNfNfNfNfNrN#fNfNf |
| | NrN#mNmGrU#rU#rU#fUfAmGmAmGmCmUmAmU#mG |
| | #mC#mU(SEQ ID NO: 22) |
| crRNA 25 | mN#mN#mN#mNmNmNmNmNmNmNfNfNfNfNrN#rN#f |
| | NfNfNmNmGrU#rU#rU#fUfAmGmAmGmCmUmAmU#m |
| | G#mC#mU(SEQ ID NO: 23) |
| crRNA 26 | mN#mN#mN#mNmNmNmNmNmNmNfNfNfNfNrN#rN#f |
| | NfNrN#mNmGfUrU#rU#fUfAmGmAmGmCmUmAmU#m |
| | G#mC#mU(SEQ ID NO: 24) |
| crRNA 27 | mN#mN#mN#mNmNmNmNmNmNmNfNfNfNfNrN#rN#f |
| | NfNrN#NAmGrU#fUrU#fUfAmGmAmGmCmUmAmU#m |
| | G#mC#mU(SEQ ID NO: 25) |
| crRNA 28 | mN#mN#mN#mNmNmNmNmNmNmNfNfNfNfNrN#rN#f |
| | NfNrN#mNmGrU#rU#rUfUfAmGmAmGmCmUmAmU#m |
| | G#mC#Mu(SEQ ID NO: 26) |
| crRNA 29 | mN#mN#mN#mNmNmNmNmNmNmNfNfNfNfNrN#fNfNf |
| | NfNmNmGfUfUfUfUfAmGmAmGmCmUmAmU#mG#mC |
| | #mU(SEQ ID NO: 27) |
| crRNA 30 | mN#mN#mN#rNrNrNmNmNmNmNrNrNrNrNrNrNrNrNrN |
| | mNmGrUrUrUrUrAmGmAmGmCmUmAmU#mG#mC#mU |
| | (SEQ ID NO: 28) |
| crRNA 31 | mN#mN#mN#rNrNrNmNmNmNmNmNrNrNrNrNrNrNrNr |
| | NmNmGrUrUrUrUrAmGmAmGmCmUmAmU#mG#mC#m |
| | U(SEQ ID NO: 29) |
| crRNA 32 | mN#mN#mN#rNrNrNmNmNmNmNmNrNmNmNrNrNrNr |
| | NrNmNmGrUrUrUrUrAmGmAmGmCmUmAmU#mG#mC |
| | #mU(SEQ ID NO: 30) |
| crRNA 33 | mN#mN#mN#rN#rN#rN#mNmNmNmNrN#rN#rN#rN# |
| | rN#rN#rN#rN#rN#mNmGrU#rU#rU#rU#rA#mGm |
| | AmGmCmUmAmU#mG#mC#mU(SEQ ID NO: 31) |
| crRNA 34 | mN#mN#mN#rN#rN#rN#mNmNmNmNrN#rN#rN#rN#rN# |
| | rN#rN#rN#rN#mNmGrUrUrUrUrAmGmAmGmCmUmAmU |
| | #mG#mC#mU(SEQ ID NO: 32) |
| crRNA 35 | mN#mN#mN#rNrNrNmNmNmNmNrNrNrNrNrNrNrNrNrN |
| | mNmGrUrUrUmUmAmGmAmGmCmUmAmU#mG#mC# |
| | mU(SEQ ID NO: 33) |
| crRNA 36 | mN#mN#mN#rN#rN#rN#mNmNmNmNrN#rN#rN#rN# |
| | rN#rN#rN#rN#rN#mNmGrUrUrUmUmAmGmAmGmCmUm |
| | AmU#mG#mC#mU(SEQ ID NO: 34) |
| crRNA 37 | mN#mN#mN#rN#rN#rN#mNmNmNmNfNfNfNfNrN#rN#f |
| | NfNrN#mNmGrU#rU#rU#mUmAmGmAmGmCmUmAmU |
| | #mG#mC#mU(SEQ ID NO: 35) |
| crRNA 38 | mN#mN#mN#dN#dN#dN#mNmNmNmNfNfNfNfNrN#rN# |
| | fNfNrN#mNmGrU#rU#rU#mUmAmGmAmGmCmUmAmU |
| | #mG#mC#mU(SEQ ID NO: 36) |
| crRNA 39 | mN#mN#mN#rN#rN#rN#mNmNmNmNfNfNfNfNfNfNfNf |
| | NfNmNmGfUfUfUfUfAmGmAmGmCmUmAmU#mG#mC |
| | #mU(SEQ ID NO: 37) |
| crRNA 40 | mN#mN#mN#dN#dN#dN#mNmNmNmNfNfNfNfNfNfNfN |
| | fNfNmNmGfUfUfUfUfAmGmAmGmCmUmAmU#mG#mC |
| | #mU(SEQ ID NO: 38) |
| crRNA 41 | mN#mN#mN#mNmNmNmNmNmNmNfNfNfNfNdN#dN#f |
| | NfNdN#mNmGrU#rU#rU#fUfAmGmAmGmCmUmAmU# |
| | mG#mC#mU(SEQ ID NO: 39) |
| crRNA 42 | mN#mN#mN#mNmNmNmNmNmNmNfNfNfNfNrN#rN#f |
| | NfNrN#mNmGdU#dU#dU#fUfAmGmAmGmCmUmAmU# |
| | mG#mC#mU(SEQ ID NO: 40) |
| crRNA 43 | mN#mN#mN#mNmNmNmNmNmNmNfNrN#fNfNrN#rN#f |
| | NfNrN#mNmGrU#rU#rU#fUfAmGmAmGmCmUmAmU# |
| | mG#mC#mU(SEQ ID NO: 41) |
| crRNA 44 | mN#mN#mN#mNmNmNmNmNmNmNfNdN#fNfNrN#rN# |
| | fNfNrN#mNmGrU#rU#rU#fUfAmGmAmGmCmUmAmU# |
| | mG#mC#mU(SEQ ID NO: 42) |
| crRNA 45 | mN#mN#mN#fNfNfNmNmNmNmNfNfNfNfNfNfNfNfN |
| | fNmNmGfUfUfUfUfAmGmAmGmCmUmAmU#mG#mC#mU |
| | (SEQ ID NO: 43) |
| crRNA 46 | mN#mN#mN#mNmNmNmNmNmNmNfNfNfNfNmNrN#f |
| | NfNrN#mNmGrU#rUrU#fUfAmGmAmGmCmUmAmU#m |
| | G#mC#mU(SEQ ID NO: 44) |
| crRNA 47 | mN#mN#mN#mNmNmNmNmNmNmNfNfNfNfNrN#mNf |
| | NfNrN#mNmGrU#rUrU#fUfAmGmAmGmCmUmAmU#m |
| | G#mC#mU(SEQ ID NO: 45) |
| crRNA 48 | mN#mN#mN#mNmNmNmNmNmNmNfNfNfNfNrN#mNf |
| | NfNmNmNmGrU#rUrU#fUfAmGmAmGmCmUmAmU#m |
| | G#mC#mU(SEQ ID NO: 46) |
| crRNA 49 | mN#mN#mN#mNmNmNmNmNmNmNfNfNfNfNrN#rN#f |
| | NfNrN#mNmGmUrU#rU#fUfAmGmAmGmCmUmAmU#m |
| | G#mC#mU(SEQ ID NO: 47) |
| crRNA 50 | mN#mN#mN#mNmNmNmNmNmNmNfNfNfNfNrN#rN#f |
| | NfNrN#mNmGrU#mUrU#fUfAmGmAmGmCmUmAmU#m |
| | G#mC#mU(SEQ ID NO: 48) |
| crRNA 51 | mN#mN#mN#mNmNmNmNmNmNmNfNfNfNfNrN#rN#f |
| | NfNrN#mNmGrU#rU#mUfUfAmGmAmGmCmUmAmU#m |
| | G#mC#mU(SEQ ID NO: 49) |
| crRNA 52 | mN#mN#mN#dN#dN#dN#mNmNmNmNfNfNfNfNfN#fN# |
| | fNfNfN#mNmGfU#fU#fU#fU#fA#mGmAmGmCmUmAm |
| | U#mG#mC#mU(SEQ ID NO: 50) |
| crRNA 53 | mN#mN#mN#dN#dN#dN#mN#mNmNmNfNfNfNfNfNfNf |
| | NfNfNmNmGfUfUfUfUfAmGmAmGmCmUmAmU#mG# |
| | mC#mU(SEQ ID NO: 51) |
| crRNA 54 | mN#mN#mN#dN#dN#dN#mNmN#mNmNfNfNfNfNfNfNf |
| | NfNfNmNmGfUfUfUfUfAmGmAmGmCmUmAmU#mG# |
| | mC#mU(SEQ ID NO: 52) |
| crRNA 55 | mN#mN#mN#dN#dN#dN#mNmNmN#mNfNfNfNfNfNfNf |
| | NfNfNmNmGfUfUfUfUfAmGmAmGmCmUmAmU#mG# |
| | mC#mU(SEQ ID NO: 53) |
| crRNA 56 | mN#mN#mN#dN#dN#dN#mNmNmNmN#fNfNfNfNfNfNf |
| | NfNfNmNmGfUfUfUfUfAmGmAmGmCmUmAmU#mG# |
| | mC#mU(SEQ ID NO: 54) |
| crRNA 57 | mN#mN#mN#dN#dN#dN#mNmNmNmNfN#fNfNfNfNfNf |
| | NfNfNmNmGfUfUfUfUfAmGmAmGmCmUmAmU#mG# |
| | mC#mU(SEQ ID NO: 55) |
| crRNA 58 | mN#mN#mN#dN#dN#dN#mNmNmNmNfNfN#fNfNfNfNf |
| | NfNfNmNmGfUfUfUfUfAmGmAmGmCmUmAmU#mG# |
| | mC#mU(SEQ ID NO: 56) |
| crRNA 59 | mN#mN#mN#dN#dN#dN#mNmNmNmNfNfNfN#fNfNfNf |
| | NfNfNmNmGfUfUfUfUfAmGmAmGmCmUmAmU#mG# |
| | mC#mU(SEQ ID NO: 57) |
| crRNA 60 | mN#mN#mN#dN#dN#dN#mNmNmNmNfNfNfNfN#fNfNf |
| | NfNfNmNmGfUfUfUfUfAmGmAmGmCmUmAmU#mG# |
| | mC#mU(SEQ ID NO: 58) |
| crRNA 61 | mN#mN#mN#dN#dN#dN#mNmNmNmNfNfNfNfNfNfNfN |
| | #fNfNmNmGfUfUfUfUfAmGmAmGmCmUmAmU#mG#m |
| | C#mU(SEQ ID NO: 59) |
| crRNA 62 | mN#mN#mN#dN#dN#dN#mNmNmNmNfNfNfNfNfNfNfN |
| | fN#fNmNmGfUfUfUfUfAmGmAmGmCmUmAmU#mG#m |
| | C#mU(SEQ ID NO: 60) |
| crRNA 63 | mN#mN#mN#dN#dN#dN#mNmNmNmNfNfNfNfNfNfNfN |
| | fNfNmN#mGfUfUfUfUfAmGmAmGmCmUmAmU#mG#m |
| | C#mU(SEQ ID NO: 61) |
| crRNA 64 | mN#mN#mN#dN#dN#dN#mNmNmNmNfNfNfNfNfNfNfN |
| | fNfNmNmG#fUfUfUfUfAmGmAmGmCmUmAmU#mG#m |
| | C#mU(SEQ ID NO: 62) |
| crRNA 65 | mN#mN#mN#dN#dN#dN#mNmNmNmNfNfNfNfNfNfNfN |
| | fNfNmNmGfUfUfUfUfAmG#mAmGmCmUmAmU#mG#m |
| | C#mU(SEQ ID NO: 63) |
| crRNA 66 | mN#mN#mN#dN#dN#dN#mNmNmNmNfNfNfNfNfNfNfN |
| | fNfNmNmGfUfUfUfUfAmGmA#mGmCmUmAmU#mG#m |
| | C#mU(SEQ ID NO: 64) |
| crRNA 67 | mN#mN#mN#dN#dN#dN#mNmNmNmNfNfNfNfNfNfNfN |
| | fNfNmNmGfUfUfUfUfAmGmAmG#mCmUmAmU#mG#m |
| | C#mU(SEQ ID NO: 65) |
| crRNA 68 | mN#mN#mN#dN#dN#dN#mNmNmNmNfNfNfNfNfNfNfN |
| | fNfNmNmGfUfUfUfUfAmGmAmGmC#mUmAmU#mG#m |
| | C#mU(SEQ ID NO: 66) |
| crRNA 69 | mN#mN#mN#dN#dN#dN#mNmNmNmNfNfNfNfNfNfNfN |
| | fNfNmNmGfUfUfUfUfAmGmAmGmCmU#mAmU#mG#m |
| | C#mU(SEQ ID NO: 67) |
| crRNA 70 | mN#mN#mN#dN#dN#dN#mNmNmNmNfNfNfNfNfNfNfN |
| | fNfNmNmGfUfUfUfUfAmGmAmGmCmUmA#mU#mG#m |
| | C#mU(SEQ ID NO: 68) |
| crRNA 71 | mN#mN#mN#dN#dN#dN#mN#mN#mN#mNfNfNfNfNfN#f |
| | N#fNfNfN#mNmGfU#fU#fU#fU#fA#mGmAmGmCmUmA |
| | mU#mG#mC#mU(SEQ ID NO: 69) |
| crRNA 72 | mN#mN#mN#dN#dN#dN#mNmNmNmN#fN#fN#fNfNfN#f |
| | N#fNfNfN#mNmGfU#fU#fU#fU#fA#mGmAmGmCmUmA |
| | mU#mG#mC#mU(SEQ ID NO: 70) |
| crRNA 73 | mN#mN#mN#dN#dN#dN#mNmNmNmNfNfNfN#fN#fN#f |
| | N#fNfNfN#mNmGfU#fU#fU#fU#fA#mGmAmGmCmUmA |
| | mU#mG#mC#mU(SEQ ID NO: 71) |
| crRNA 74 | mN#mN#mN#dN#dN#dN#mNmNmNmNfNfNfNfNfN#fN# |
| | fN#fN#fN#mNmGfU#fU#fU#fU#fA#mGmAmGmCmUmA |
| | mU#mG#mC#mU(SEQ ID NO: 72) |
| crRNA 75 | mN#mN#mN#dN#dN#dN#mNmNmNmNfNfNfNfNfN#fN# |
| | fNfNfN#mN#mGfU#fU#fU#fU#fA#mGmAmGmCmUmAm |
| | U#mG#mC#mU(SEQ ID NO: 73) |
| crRNA 76 | mN#mN#mN#dN#dN#dN#mN#mN#mN#mNfNfNfNfNfNf |
| | NfNfNfNmNmGfUfUfUfUfAmGmAmGmCmUmAmU#mG |
| | #mC#mU(SEQ ID NO: 74) |
| crRNA 77 | mN#mN#mN#dN#dN#dN#mN#mN#mN#mN#fN#fN#fNfNf |
| | NfNfNfNfNmNmGfUfUfUfUfAmGmAmGmCmUmAmU# |
| | mG#mC#mU(SEQ ID NO: 75) |
| crRNA 78 | mN#mN#mN#dN#dN#dN#mN#mN#mN#mN#fN#fN#fN#f |
| | N#fN#fN#fNfNfNmNmGfUfUfUfUfAmGmAmGmCmUmA |
| | mU#mG#mC#mU(SEQ ID NO: 76) |
| crRNA 79 | mN#mN#mN#dN#dN#dN#mN#mN#mN#mN#fN#fN#fN#f |
| | N#fN#fN#fN#fN#fN#mNmGfUfUfUfUfAmGmAmGmCmU |
| | mAmU#mG#mC#mU(SEQ ID NO: 77) |
| crRNA 80 | mN#mN#mN#dN#dN#dN#mN#mN#mN#mN#fN#fN#fN#f |
| | N#fN#fN#fN#fN#fN#mN#mG#fU#fU#fU#fU#fA#mGm |
| | GAmmCmUmAmU#mG#mC#mU(SEQ ID NO: 78) |
| crRNA 81 | mN#mN#mN#dN#dN#dN#mNmNmNmNfNfNfNfNfN#fN# |
| | fNfNfN#mNmGfU#fU#fU#fU#fA#mGmAmGmC#mU#mA |
| | #mU#mG#mC#mU(SEQ ID NO: 79) |
| crRNA 82 | mN#mN#mN#dN#dN#dN#mNmNmNmNfNfNfNfNfN#fN# |
| | fNfNfN#mNmGfU#fU#fU#fU#fA#mG#mA#mG#mCmUm |
| | AmU#mG#mC#mU(SEQ ID NO: 80) |
| crRNA 83 | mN#mN#mN#dN#dN#dN#mNmNmNmNfNfNfNfNfN#fN# |
| | fNfNfN#mNmG#fU#fU#fU#fU#fA#mGmAmGmCmUmAm |
| | U#mG#mC#mU(SEQ ID NO: 81) |
| crRNA 84 | mN#mN#mN#dN#dN#dN#mNmNmNmNfNfNfNfNfNfNfN |
| | fNfNmNmGfUfUfUfUfAmGmAmGmC#mU#mA#mU#mG |
| | #mC#mU(SEQ ID NO: 82) |
| crRNA 85 | mN#mN#mN#dN#dN#dN#mNmNmNmNfNfNfNfNfNfNfN |
| | fNfNmNmGfUfUfUfUfA#mG#mA#mG#mC#mU#mA#mU |
| | #mG#mC#mU(SEQ ID NO: 83) |
| crRNA 86 | mN#mN#mN#dN#dN#dN#mNmNmNmNfNfNfNfNfNfNfN |
| | fNfNmNmGfU#fU#fU#fU#fA#mG#mA#mG#mC#mU#mA |
| | #mU#mG#mC#mU(SEQ ID NO: 84) |
| crRNA 87 | mN#mN#mN#dN#dN#dN#mN#mN#mNmNfNfNfNfNfNfN |
| | fNfNfNmNmGfUfUfUfUfAmGmAmGmCmU#mA#mU#m |
| | G#mC#mU(SEQ ID NO: 85) |
| crRNA 88 | mN#mN#mN#dN#dN#dN#mN#mN#mN#mN#fNfNfNfNfNf |
| | NfNfNfNmNmGfUfUfUfUfAmGmAmG#mC#mU#mA#mU |
| | #mG#mC#mU(SEQ ID NO: 86) |
| crRNA 89 | mN#mN#mN#dN#dN#dN#mN#mN#mN#mN#fN#fN#fNfNf |
| | NfNfNfNfNmNmGfUfUfUfUfAmG#mA#mG#mC#mU#mA |
| | #mU#mG#mC#mU(SEQ ID NO: 87) |
| crRNA 90 | mN#mN#mN#dN#dN#dN#mN#mN#mN#mN#fN#fN#fN#f |
| | N#fNfNfNfNfNmNmGfUfUfUfU#fA#mG#mA#mG#mC#m |
| | U#mA#mU#mG#mC#mU(SEQ ID NO: 88) |
| crRNA 91 | mN#mN#mN#dN#dN#dN#mN#mN#mN#mN#fN#fN#fN#f |
| | N#fN#fN#fNfNfNmNmGfUfU#fU#fU#fA#mG#mA#mG# |
| | mC#mU#mA#mU#mG#mC#mU(SEQ ID NO: 89) |
| crRNA 92 | mN#mN#mN#dN#dN#dN#mN#mN#mN#mN#fN#fN#fN#f |
| | N#fN#fN#fN#fN#fN#mNmGfU#fU#fU#fU#fA#mG#m |
| | A#mG#mC#mU#mA#mU#mG#mC#mU(SEQ ID NO: 90) |
| crRNA 93 | mN#mN#mN#dN#dN#dN#mN#mN#mN#mN#fN#fN#fN#f |
| | N#fN#fN#fN#fN#fN#mN#mG#fU#fU#fU#fU#fA#mG# |
| | mA#mG#mC#mU#mA#mU#mG#mC#mU(SEQ ID NO: 91) |
| crRNA 94 | mN#mN#mN#mN#mN#mN#mNmNmNmNfNfNfNfNrN#r |
| | N#fNfNrN#mNmGrU#rU#rU#fUfAmGmAmGmCmUmAm |
| | U#mG#mC#mU(SEQ ID NO: 92) |
| crRNA 95 | mN#mN#mN#mN#mN#mN#mN#mNmNmNfNfNfNfNrN#r |
| | N#fNfNrN#mNmGrU#rU#rU#fUfAmGmAmGmCmUmAm |
| | U#mG#mC#mU(SEQ ID NO: 93) |
| crRNA 96 | mN#mN#mN#mN#mN#mN#mNmNmNmNfNfNfNfNrN#r |
| | N#fNfNrN#mNmGrU#rU#rU#fUfAmGmAmGmC#mUmA |
| | mU#mG#mC#mU(SEQ ID NO: 94) |
| crRNA 97 | mN#mN#mN#mN#mN#mN#mNmNmNmNfNfNfNfNrN#r |
| | N#fNfNrN#mNmGrU#rU#rU#fUfAmGmAmGmCmU#mA |
| | mU#mG#mC#mU(SEQ ID NO: 95) |
| crRNA 98 | mN#mN#mN#mN#mN#mN#mNmNmNmNfNfNfNfNrN#r |
| | N#fNfNrN#mGNmGrU#rU#rU#fUfAmGmAmGmCmUmA |
| | #mU#mG#mC#mU(SEQ ID NO: 96) |
| crRNA 99 | mN#mN#mN#mN#mN#mN#mNmNmNmNfNfNfNfNrN#r |
| | N#fNfNrN#mNmGrU#rU#rU#fU#fA#mGmAmGmCmUmA |
| | mU#mG#mC#mU(SEQ ID NO: 97) |
| crRNA 100 | mN#mN#mN#mN#mN#mN#mNmNmNmNfNfNfNfNrN#r |
| | N#fNfNrN#mNmGrU#rU#rU#fU#fA#mGmAmGmC#mU# |
| | mA#mU#mG#mC#mU(SEQ ID NO: 98) |
| crRNA 101 | mN#mN#mN#mN#mN#mN#mNmNmNmNfNfNfNfNrN#r |
| | N#fNfNrN#mNmGrU#rU#rU#fU#fA#mG#mA#mG#mCmU |
| | mAmU#mG#mC#mU(SEQ ID NO: 99) |
| crRNA 102 | mN#mN#mN#mN#mN#mN#mNmNmNmNfNfNfNfNrN#r |
| | N#fNfNrN#mNmG#rU#rU#rU#fU#fA#mGmAmGmCmUm |
| | AmU#mG#mC#mU(SEQ ID NO: 100) |
| crRNA 103 | mN#mN#mN#mN#mN#mN#mNmNmNmNfNfNfNfNrN#r |
| | N#fNfNrN#mNmGrU#rU#rU#fUfAmGmAmGmC#mU#mA |
| | #mU#mG#mC#mU(SEQ ID NO: 101) |
| crRNA 104 | mN#mN#mN#mN#mN#mN#mNmNmNmNfNfNfNfNrN#r |
| | N#fNfNrN#mNmGrU#rU#rU#fUfA#mG#mA#mG#mC#mU |
| | #mA#mU#mG#mC#mU(SEQ ID NO: 102) |
| crRNA 105 | mN#mN#mN#rN#rN#rN#mN#mNmNmNfNfNfNfNfNfNfN |
| | fNfNmNmGfUfUfUfUfAmGmAmGmCmUmAmU#mG#mC |
| | #mU(SEQ ID NO: 103) |
| crRNA 106 | mN#mN#mN#rN#rN#rN#mNmNmNmNfNfNfNfNfNfNfNf |
| | NfNmNmGfUfUfUfUfAmGmAmGmCmUmA#mU#mG#m |
| | C#mU(SEQ ID NO: 104) |
| crRNA 107 | mN#mN#mN#rN#rN#rN#mNmNmNmNfNfNfNfNfN#fN#f |
| | N#fN#fN#mNmGfU#fU#fU#fU#fA#mGmAmGmCmUmA |
| | mU#mG#mC#mU(SEQ ID NO: 105) |
| crRNA 108 | mN#mN#mN#rN#rN#rN#mNmNmNmNfNfNfNfNfN#fN#f |
| | NfNfN#mNmGfU#fU#fU#fU#fA#mGmAmGmC#mU#mA# |
| | mU#mG#mC#mU(SEQ ID NO: 106) |
| crRNA 109 | mN#mN#mN#rN#rN#rN#mNmNmNmNfNfNfNfNfN#fN#f |
| | NfNfN#mNmGfU#fU#fU#fU#fA#mG#mA#mG#mCmUmA |
| | mU#mG#mC#mU(SEQ ID NO: 107) |
| crRNA 110 | mN#mN#mN#rN#rN#rN#mNmNmNmNfNfNfNfNfN#fN#f |
| | NfNfN#mNmG#fU#fU#fU#fU#fA#mGmAmGmCmUmAm |
| | U#mG#mC#mU(SEQ ID NO: 108) |
| crRNA 111 | mN#mN#mN#rN#rN#rN#mNmNmNmNfNfNfNfNfNfNfNf |
| | NfNmNmGfUfUfUfUfAmGmAmGmC#mU#mAmU#mG#m |
| | C#mU(SEQ ID NO: 109) |
| crRNA 112 | mN#mN#mN#rN#rN#rN#mNmNmNmNfNfNfNfNfNfNfNf |
| | NfNmNmGfUfUfUfUfA#mG#mA#mG#mC#mU#mAmU#m |
| | G#mC#mU(SEQ ID NO: 110) |
| crRNA 113 | mN#mN#mN#dN#dN#dN#mNmNmNmNfNfNfNfNdN#dN |
| | #fNfNdN#mNmGdU#dU#dU#fUfAmGmAmGmCmUmAm |
| | U#mG#mC#mU(SEQ ID NO: 111) |
| crRNA 114 | mN#mN#mN#mNmNmNmNmNmNmNfNfNfNfNrN#rN#f |
| | NfNaNmNmGaUaUaUfUfAmGmAmGmCmUmAmU#mG# |
| | mC#mU(SEQ ID NO: 112) |
| crRNA 115 | mN#mN#mN#mNmNmNmNmNmNmNfNfNfNfNrN#rN#f |
| | NfNaNmNmGrU#rU#rU#fUfAmGmAmGmCmUmAmU#m |
| | G#mC#mU(SEQ ID NO: 113) |
| crRNA 116 | mN#mN#mN#mNmNmNmNmNmNmNfNfNfNfNrN#rN#f |
| | NfNrN#mNmGaUrU#rU#fUfAmGmAmGmCmUmAmU#m |
| | G#mC#mU(SEQ ID NO: 114) |
| crRNA 117 | mN#mN#mN#mNmNmNmNmNmNmNfNfNfNfNrN#rN#f |
| | NfNrN#mNmGrU#aUrU#fUfAmGmAmGmCmUmAmU#m |
| | G#mC#mU(SEQ ID NO: 115) |
| crRNA 118 | mN#mN#mN#mNmNmNmNmNmNmNfNfNfNfNrN#rN#f |
| | NfNrN#mNmGrU#rU#aUfUfAmGmAmGmCmUmAmU#m |
| | G#mC#mU(SEQ ID NO: 116) |
| crRNA 119 | mN#mN#mN#mNmNmNmNmNmNmNfNfNfNfNrN#rN#f |
| | NfNsN#mNmGsU#sU#sU#fUfAmGmAmGmCmUmAmU# |
| | mG#mC#mU(SEQ ID NO: 117) |
| crRNA 120 | mN#mN#mN#mNmNmNmNmNmNmNfNfNfNfNrN#rN#f |
| | NfNsNmNmGsUsUsUfUfAmGmAmGmCmUmAmU#mG# |
| | mC#mU(SEQ ID NO: 118) |
| crRNA 121 | mN#mN#mN#mNmNmNmNmNmNmNfNfNfNfNrN#rN#f |
| | NfNsNmNmGrU#rU#rU#fUfAmGmAmGmCmUmAmU#m |
| | G#mC#mU(SEQ ID NO: 119) |
| crRNA 122 | mN#mN#mN#mNmNmNmNmNmNmNfNfNfNfNrN#rN#f |
| | NfNrN#mNmGsUrU#rU#fUfAmGmAmGmCmUmAmU#m |
| | G#mC#mU(SEQ ID NO: 120) |
| crRNA 123 | mN#mN#mN#mNmNmNmNmNmNmNfNfNfNfNrN#rN#f |
| | NfNrN#mNmGrU#sUrU#fUfAmGmAmGmCmUmAmU#m |
| | G#mC#mU(SEQ ID NO: 121) |
| crRNA 124 | mN#mN#mN#mNmNmNmNmNmNmNfNfNfNfNrN#rN#f |
| | NfNrN#mNmGrU#rU#sUfUfAmGmAmGmCmUmAmU#m |
| | G#mC#mU(SEQ ID NO: 122) |
| crRNA 125 | mN#mN#mN#mNmNmNmNmNmNmNfNfNfNfNrN#rN#f |
| | NfNrN#mNmGsUrU#sUfUfAmGmAmGmCmUmAmU#mG |
| | #mC#mU(SEQ ID NO: 123) |
| crRNA 126 | mN#mN#mN#mNmNmNmNmNmNmNfNfNfNfNrN#rN#f |
| | NfNrN#mNmGsUsUrU#fUfAmGmAmGmCmUmAmU#mG |
| | #mC#mU(SEQ ID NO: 124) |
| crRNA 127 | mN#mN#mN#mNmNmNmNmNmNmNfNfNfNfNrN#rN#f |
| | NfNrN#mNmGrU#sUsUfUfAmGmAmGmCmUmAmU#mG |
| | #mC#mU(SEQ ID NO: 125) |
| crRNA 128 | mN#mN#mN#mNmNmNmNmNmNmNfNfNfNfNrN#rN#f |
| | NfNrN#mNmGaUaUaUfUfAmGmAmGmCmUmAmU#mG |
| | #mC#mU(SEQ ID NO: 126) |
| crRNA 129 | mN#mN#mN#mNmNmNmNmNmNmNfNfNfNfNrN#rN#f |
| | NfNrN#mNmGsU#sU#sU#fUfAmGmAmGmCmUmAmU# |
| | mG#mC#mU (SEQ ID NO: 127) |
| crRNA 130 | mN#mN#mN#mNmNmNmNmNmNmNfNfNfNfNrN#rN#f |
| | NfNrN#mNmGsUsUsUfUfAmGmAmGmCmUmAmU#mG# |
| | mC#mU (SEQ ID NO: 128) |
| crRNA 131 | mN#mN#mN#mNmNmNmNmNmNmNfNfNfNfNrN#aNfN |
| | fNrN#mNmGrU#rU#rU#fUfAmGmAmGmCmUmAmU#m |
| | G#mC#mU (SEQ ID NO: 129) |
| crRNA 132 | mN#mN#mN#mNmNmNmNmNmNmNfNfNfNfNrN#rN#f |
| | NfNaNmNmGrU#rU#rU#fUfAmGmAmGmCmUmAmU#m |
| | G#mC#mU(SEQ ID NO: 113) |
| crRNA 133 | mN#mN#mN#mNmNmNmNmNmNmNfNfNfNfNrN#aN#f |
| | NfNaN#mNmGrU#rU#rU#fUfAmGmAmGmCmUmAmU# |
| | mG#mC#mU (SEQ ID NO: 130) |
| crRNA 134 | mN#mN#mN#mNmNmNmNmNmNmNfNfNfNfNrN#rN#f |
| | NfNrN#mNmGaUaUaUfUfAmGmAmGmCmUmAmU#mG |
| | #mC#mU(SEQ ID NO: 126) |

KEY:
rN = RNA,
mN = 2′-O-methyl RNA,
fN = 2′-fluoro RNA,
aN = 2′-NH₂ (2′-amino RNA),
sN = 4′-thio RNA,
dN = 2′-deoxy RNA
N = A, U, G, or C
N#N = phosphorothioate linkage

It will be understood to those of skill in the art that the base sequence of the first 20 nucleotides of the exemplary crRNAs recited in Table 2 above are directed to a specific target. This 20-nucleotide base sequence may be changed based on the target nucleic acid, however the chemical modifications remain the same. An exemplary unmodified crRNA sequence, from 5′ to 3′, is

(SEQ ID NO: 1)
NNNNNNNNNNNNNNNNNNNNGUUUUAGAGCUAUGCU,

where “N” corresponds to any nucleotide (e.g., A, U, G, or C). An exemplary unmodified tracrRNA sequence, from 5′ to 3′, is

(SEQ ID NO: 2)
AGCAUAGCAAGUUAAAAUAAGGCUAGUCCGUUAUC
AACUUGAAAAAGUGGCACCGAGUCGGUGCUUU.

It will be further understood to those of skill in the art that the guide sequence may be 10-30 nucleotides in length, preferably 16-24 nucleotides in length (for example, 16, 17, 18, 19, 20, 21, 22, 23 or 24 nucleotides in length), and is at or near the 5′ terminus of a Cas9 gRNA.

Chemically modified guide RNAs, including chemically modified crRNAs and tracrRNAs, are described in more detail in US20210363518 and US20210388348, each of which is incorporated herein by reference.

The protecting oligonucleotides described herein may be designed to bind to any region of the crRNA modification patterns that contain at least one unmodified ribose group, thereby conferring nuclease resistance to the crRNA.

### High-Affinity Repeat/Anti-Repeat Guide RNA Modifications

A crRNA and a tracrRNA hybridize together by forming a duplex between the repeat region of the crRNA and the anti-repeat region of the tracrRNA. In certain embodiments, modular, or dual RNA, guide RNAs are provided with modifications in the repeat region and the anti-repeat region to enhance the affinity between the two regions and form a stronger duplex.

The high-affinity interaction may be enhanced by increasing the GC nucleotide content in the duplex formed by the repeat regions and the anti-repeat region. Nucleotide modifications, such as 2′-Fluoro and 2′-O-Methyl modifications, may also be introduced, which increase the melting temperature (Tm) of the duplex. Further modifications include the use of orthogonal and non-naturally occurring nucleotides. The various repeat region/anti-repeat region modifications described herein enhance the stability of the duplex, helping to prevent the crRNA and tracrRNA from folding into sub-optimal structures and therefore promoting higher genome editing efficacy.

### Conjugates

The protecting oligonucleotides and/or crRNAs of the disclosure may be modified with terminally conjugated moieties. As used herein, a “terminally conjugated moiety” or “moiety” refers to a compound which may be linked or attached to the 5′ and/or 3′ end of the crRNA and/protecting oligonucleotide. Terminally conjugated moieties can provide increased stability, increased ability to penetrate cell membranes, increase cellular uptake, increase circulation time in vivo, act as a cell-specific directing reagent, and/or provide a means to monitor cellular or tissue-specific uptake.

In certain embodiments, the terminally conjugated moiety is conjugated to the 5′ end of the crRNA. In certain embodiments, the terminally conjugated moiety is conjugated to the 3′ end of the crRNA. In certain embodiments, the terminally conjugated moiety is conjugated to the 5′ end of the protecting oligonucleotide. In certain embodiments, the terminally conjugated moiety is conjugated to the 3′ end of the protecting oligonucleotide.

In certain exemplary embodiments, a terminally conjugated moiety includes, but is not limited to, fatty acid, steroid, secosteroid, lipid, ganglioside analog, nucleoside analogs, endocannabinoid, vitamin, receptor ligand, peptide, aptamer, alkyl chain, fluorophore, antibody, nuclear localization signal, and the like.

In certain exemplary embodiments, a terminally conjugated moiety includes, but is not limited to, cholesterol, cholesterol-triethylene glycol (TEGChol), docosahexaenoic acid (DHA), docosanoic acid (DCA), lithocholic acid (LA), GalNAc, amphiphilic block copolymer (ABC), hydrophilic block copolymer (HBC), poloxamer, Cy5, Cy3, and the like.

In certain exemplary embodiments, the at least one terminally conjugated moiety is a modified lipid, including a branched lipid (such as the structure shown in Formula I) or a headgroup-modified lipid (such as the structure shown in Formula II).

X-MC(═Y)M-Z-[L-MC(═Y)M-R]n Formula I

where X is a moiety that links the lipid to the guide RNA, each Y is independently oxygen or sulfur, each M is independently CH₂, NH, O or S, Z is a branching group which allows two or three (“n”) chains to be joined to the rest of the structure, L is an optional linker moiety, and each R is independently a saturated, monounsaturated or polyunsaturated linear or branched moiety from 2 to 30 atoms in length, a sterol, or other hydrophobic group.

X-MC(═Y)M-Z-[L-MC(═Y)M-R]n-L-K-J Formula II

where X is a moiety that links the lipid to the guide RNA, each Y is independently oxygen or sulfur, each M is independently CH₂, NH, N-alkyl, O or S, Z is a branching group which allows two or three (“n”) chains to be joined to the rest of the structure, each L is independently an optional linker moiety, and R is a saturated, monounsaturated or polyunsaturated linear or branched moiety from 2 to 30 atoms in length, a sterol, or other hydrophobic group, K is a phosphate, sulfate, or amide and J is an aminoalkane or quaternary aminoalkane group.

The moieties may be attached to the terminal nucleotides of the guide RNA via a linker. Exemplary linkers include, but are not limited to, an ethylene glycol chain, an alkyl chain, a polypeptide, a polysaccharide, a block copolymer, and the like.

In certain embodiments, the moiety is conjugated to the 5′ end and/or 3′ end of any one of RC01 to RC10 (i.e., RC01, RC02, RC03, RC04, RC05, RC06, RC07, RC08, RC09, or RC10).

### Chemically Modified Single Guide RNA

As described herein, the chemically modified guide RNAs of the disclosure may be constructed as single guide RNAs (sgRNAs) by linking the 3′ end of a crRNA to the 5′ end of a tracrRNA. The linker may be an oligonucleotide loop, including a chemically modified oligonucleotide loop. In certain embodiments, the oligonucleotide loop comprises a GAAA tetraloop. The linker may be a non-nucleotide chemical linker, including, but not limited to, ethylene glycol oligomers (see, e.g., Pils et al. Nucleic Acids Res. 28(9): 1859-1863 (2000)).

### RNA-Guided Nucleases

RNA-guided nucleases according to the present disclosure include, without limitation, naturally-occurring Type II CRISPR nucleases such as Cas9, as well as other nucleases derived or obtained therefrom. Exemplary Cas9 nucleases that may be used in the present disclosure include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9). In functional terms, RNA-guided nucleases are defined as those nucleases that: (a) interact with (e.g., complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g., Cas9 vs. Cpf1), species (e.g., S. pyogenes vs. S. aureu ) or variation (e.g., full-length vs. truncated or split; naturally-occurring PAM specificity vs. engineered PAM specificity).

Various RNA-guided nucleases may require different sequential relationships between PAMs and protospacers. In general, Cas9s recognize PAM sequences that are 5′ of the protospacer as visualized relative to the top or complementary strand.

In addition to recognizing specific sequential orientations of PAMs and protospacers, RNA-guided nucleases generally recognize specific PAM sequences. S. aureus Cas9, for example, recognizes a PAM sequence of NNGRRT, wherein the N sequences are immediately 3′ of the region recognized by the gRNA targeting domain. S. pyogenes Cas9 recognizes NGG PAM sequences. It should also be noted that engineered RNA-guided nucleases can have PAM specificities that differ from the PAM specificities of similar nucleases (such as the naturally occurring variant from which an RNA-guided nuclease is derived, or the naturally occurring variant having the greatest amino acid sequence homology to an engineered RNA-guided nuclease). Modified Cas9s that recognize alternate PAM sequences are described below.

RNA-guided nucleases are also characterized by their DNA cleavage activity: naturally-occurring RNA-guided nucleases typically form DSBs in target nucleic acids, but engineered variants have been produced that generate only SSBs (discussed above; see also Ran 2013, incorporated by reference herein), or that do not cut at all.

The RNA-guided nuclease Cas9 may be a variant of Cas9 with altered activity. Exemplary variant Cas9 nucleases include, but are not limited to, a Cas9 nickase (nCas9), a catalytically dead Cas9 (dCas9), a hyper accurate Cas9 (HypaCas9) (Chen et al. Nature, 550(7676), 407-410 (2017)), a high fidelity Cas9 (Cas9-HF) (Kleinstiver et al. Nature 529(7587), 490-495 (2016)), an enhanced specificity Cas9 (eCas9) (Slaymaker et al. Science 351(6268), 84-88 (2016)), and an expanded PAM Cas9 (xCas9) (Hu et al. Nature doi: 10.1038/nature26155 (2018)).

The RNA-guided nucleases may be combined with the chemically modified guide RNAs of the present disclosure to form a genome-editing system. The RNA-guided nucleases may be combined with the chemically modified guide RNAs to form an RNP complex that may be delivered to a cell where genome-editing is desired. The RNA-guided nucleases may be expressed in a cell where genome-editing is desired with the chemically modified guide RNAs delivered separately. For example, the RNA-guided nucleases may be expressed from a polynucleotide such as a vector or a synthetic mRNA. The vector may be a viral vector, including, be not limited to, an adeno-associated virus (AAV) vector or a lentivirus (LV) vector.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Genome Editing Efficiency of Chemically Modified crRNA and tracrRNA

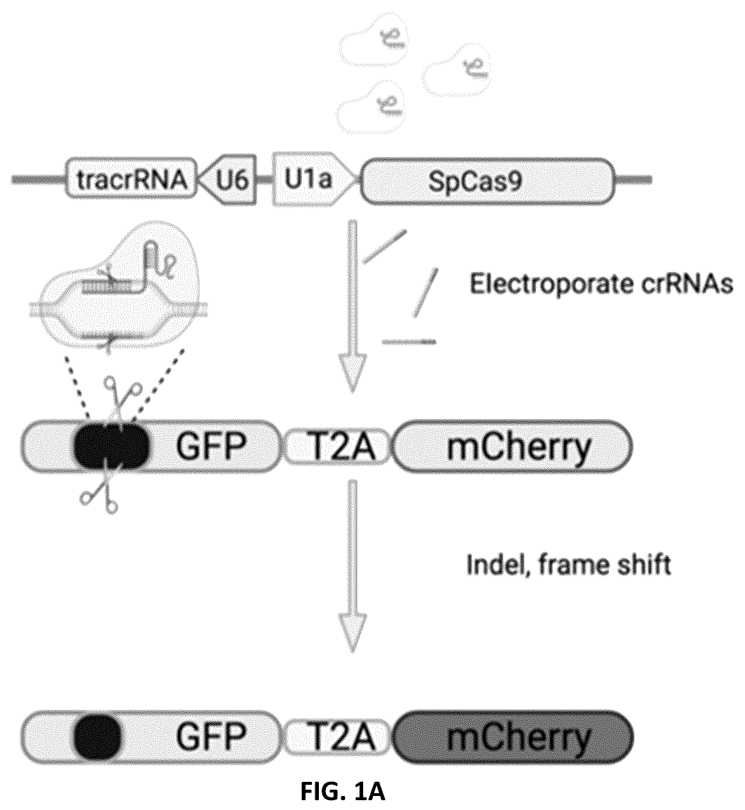

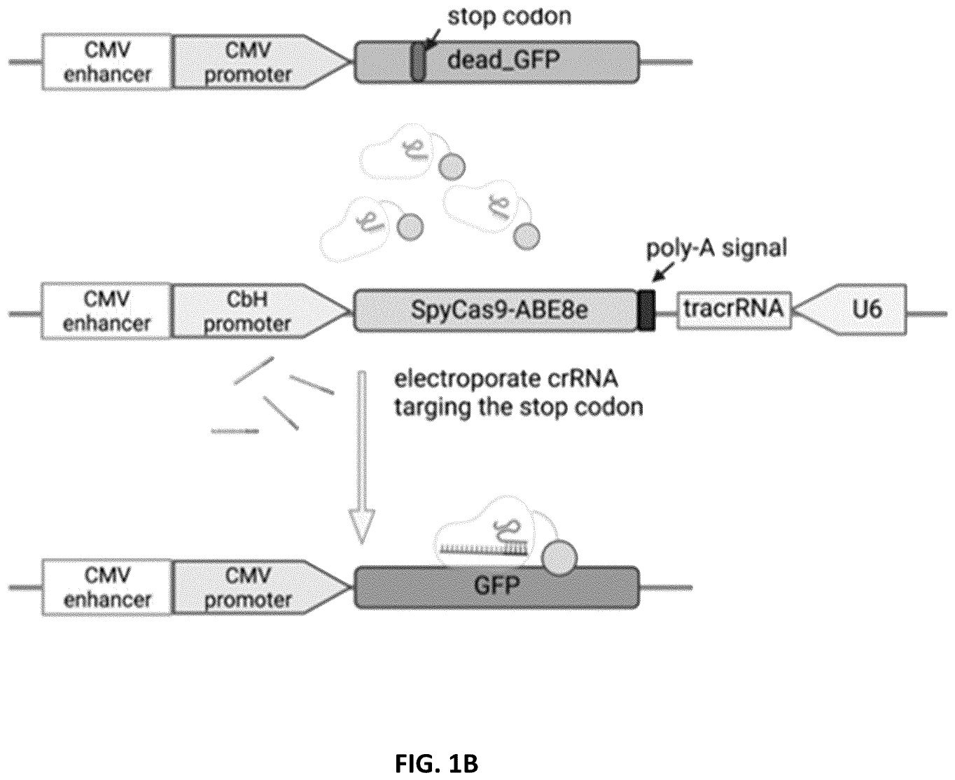

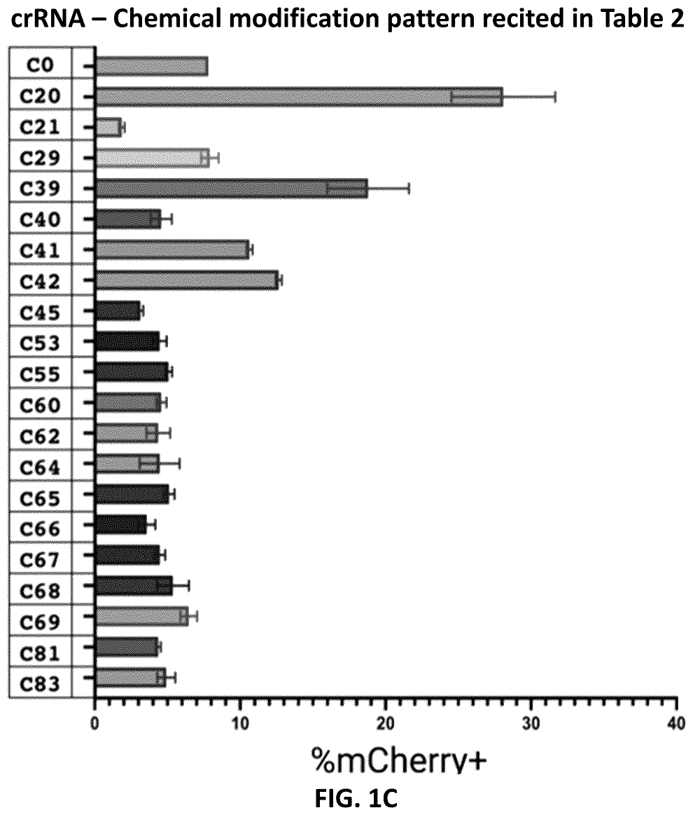

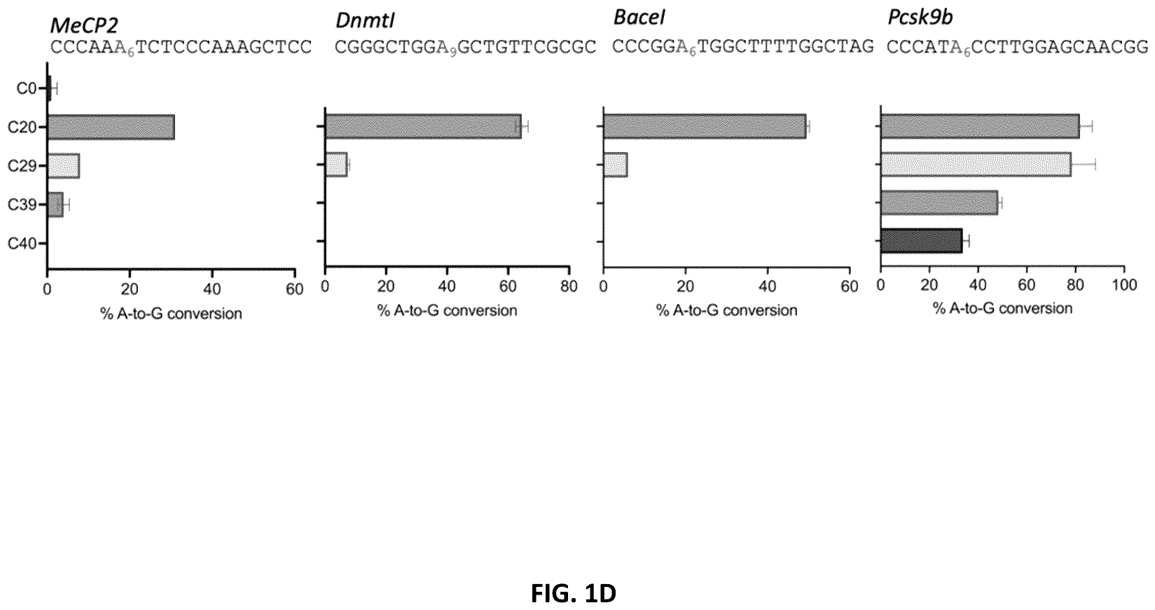

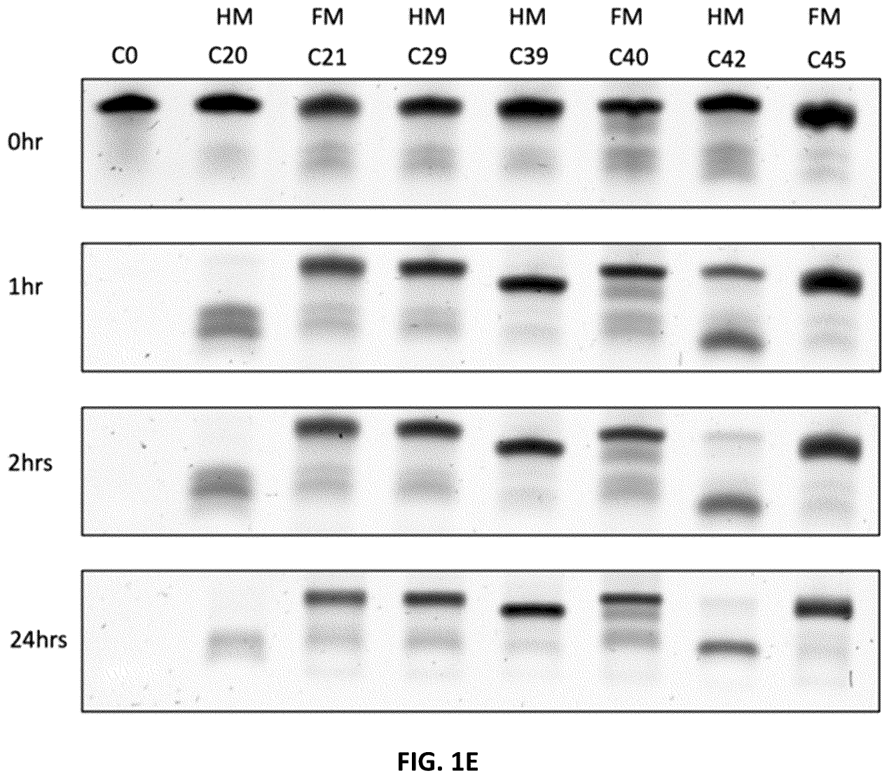

To test the compatibility between a certain chemical modification of the crRNA and SpyCas9/base editor (BE) activity, we established a scenario in which cell lines stably express either SpyCas9 nuclease or base editor, tracrRNA, and a fluorescent reporter gene (FIG. 1A and FIG. 1B), but do not express a crRNA. We first screened the activities of different chemically modified crRNAs designed previously (described in WO 2019/183000 A1 and WO 2021/231606 A2, incorporated herein by reference) by electroporation into the stable cell lines and quantifying the percentage of fluorescence-positive cells (which reports on editing activity) using fluorescent activated cell sorting (FACS). Compared to the end-modified C0 crRNA, the heavily modified C20 showed significantly enhanced activity, while all the fully modified crRNAs showed reduced activity (FIG. 1C). This conclusion was further confirmed using crRNAs targeting various endogenous genomic loci (FIG. 1D). We next compared the stabilities of the heavily and fully modified crRNAs by incubating them with fetal bovine serum (FBS). We found that only the fully modified crRNAs are stable in the FBS (FIG. 1E). We note that C20 has six ribose residues that include a 3′-phosphorothioate; phosphorothioate modifications improves stability relative to unmodified phosphate linkages, but substantial degradation does still occur. Therefore, our most active CRISPR guide (C20) requires further stabilization.

The protecting oligonucleotides used in the Examples are recited below in Table 3. Table 4 below recites crRNAs used in the Examples.

TABLE 3
Exemplary Protecting Oligonucleotides

| Name | Sequence |
|---|---|
| RC04_MCV1 | mAmAmAmAmCmCmGmAmGmGmCmA(SEQ ID |
| a | NO: 131) |
| RC05_MCV1 | mUmAmAmAmAmCmCmGmAmGmGmCmA(SEQ ID |
| a | NO: 132) |
| RC06_MCV1 | mAmAmAmAmCmCmGmAmGmGmCmAmG(SEQ ID |
| a | NO: 133) |
| RC07_MCV1 | mUmAmAmAmAmCmCmGmAmGmGmCmAmG(SEQ ID |
| a | NO: 134) |
| RC08_MCV1 | mCmUmAmAmAmAmCmCmGmAmGmGmCmAmG(SEQ |
| a | ID NO: 135) |
| RC09_MCV1 | mUmAmAmAmAmCmCmGmAmGmGmCmAmGmG(SEQ |
| a | ID NO: 136) |
| RC10_MCV1 | mCmUmAmAmAmAmCmCmGmAmGmGmCmAmGmG(S |
| a | EQ ID NO: 137) |
| RC07_MCV1 | mUmAmAmAmAmCmCmGmAmGmGmCmAmG- |
| a-TegChol | TegChol(SEQ ID NO: 134) |
| RC07_MCV1 | mUmAmAmAmAmCmCmGmAmGmGmCmAmGdTdT- |
| a-dT- | TegChol(SEQ ID NO: 138) |
| TegChol | |
| RC07_MCV1 | lUlAmAmAmAmCmCmGmAmGmGmClAlG(SEQ ID |
| a-4xLNA | NO: 139) |
| RC07_MCV1 | lUmAmAmAmAmCmCmGmAmGmGmCmAlG(SEQ ID |
| a-2xLNA | NO: 140) |
| Cy5- | Cy5-mUmAmAmAmAmCmCmGmAmGmGmCmAmG- |
| RC07_MCV1 | TegChol(SEQ ID NO: 134) |
| a-TegChol | |
| RC07_dGFP | mUmAmAmAmAmCmAmCmCmAmCmAmUmG(SEQ ID |
| | NO: 141) |
| RC08_dGFP | mCmUmAmAmAmAmCmAmCmCmAmCmAmUmG |
| | (SEQ ID NO: 142) |
| RC09_dGFP | mUmAmAmAmAmCmAmCmCmAmCmAmUmGmA |
| | (SEQ ID NO: 143) |
| RC10_dGFP | mCmUmAmAmAmAmCmAmCmCmAmCmAmUmGmA |
| | (SEQ ID NO: 144) |
| RC07- | mUmAmAmAmAmCmAmCmCmAmCmAmUmG- |
| GalNac_dGF | GalNac(SEQ ID NO: 141) |
| P | |
| RC08- | mCmUmAmAmAmAmCmAmCmCmAmCmAmUmG- |
| GalNac_dGF | GalNac(SEQ ID NO: 142) |
| P | |
| RC09- | mUmAmAmAmAmCmAmCmCmAmCmAmUmGmA- |
| GalNac_dGF | GalNac(SEQ ID NO: 143) |
| P | |
| RC10- | mCmUmAmAmAmAmCmAmCmCmAmCmAmUmGmA- |
| GalNac_dGF | GalNac(SEQ ID NO: 144) |
| P | |
| RC07- | mUmAmAmAmAmCmAmCmCmAmCmAmUmG- |
| DHA_dGFP | DHAvl (SEQ ID NO: 141) |
| RC08- | mCmUmAmAmAmAmCmAmCmCmAmCmAmUmG- |
| DHA_dGFP | DHAvl (SEQ ID NO: 142) |
| RC09- | mUmAmAmAmAmCmAmCmCmAmCmAmUmGmA- |
| DHA_dGFP | DHAvl (SEQ ID NO: 143) |
| RC10- | mCmUmAmAmAmAmCmAmCmCmAmCmAmUmGmA- |
| DHA_dGFP | DHAvl (SEQ ID NO: 144) |
| RC07-dT- | mUmAmAmAmAmCmAmCmCmAmCmAmUmGdTdT- |
| DHA_dGFP | DHAvl (SEQ ID NO: 145) |
| RC08-dT- | mCmUmAmAmAmAmCmAmCmCmAmCmAmUmGdTdT- |
| DHA_dGFP | DHAvl (SEQ ID NO: 146) |
| RC09-dT- | mUmAmAmAmAmCmAmCmCmAmCmAmUmGmAdTdT |
| DHA_dGFP | -DHAvl (SEQ ID NO: 147) |
| RC10-dT- | mCmUmAmAmAmAmCmAmCmCmAmCmAmUmGmAd |
| DHA_dGFP | TdT-DHAvl (SEQ ID NO: 148) |
| RC07- | mUmAmAmAmAmCmAmCmCmAmCmAmUmG-TegChol |
| TegChol_dG | (SEQ ID NO: 141) |
| FP | |
| RC08- | mCmUmAmAmAmAmCmAmCmCmAmCmAmUmG- |
| TegChol_dG | TegChol(SEQ ID NO: 142) |
| FP | |
| RC07-dT- | mUmAmAmAmAmCmAmCmCmAmCmAmUmGdTdT- |
| TegChol_dG | TegChol(SEQ ID NO: 145) |
| FP | |
| RC08-dT- | mCmUmAmAmAmAmCmAmCmCmAmCmAmUmGdTdT- |
| TegChol_dG | TegChol(SEQ ID NO: 146) |
| FP | |
| RC09-dT- | mUmAmAmAmAmCmAmCmCmAmCmAmUmGmAdTdT |
| TegChol_dG | -TegChol(SEQ ID NO: 147) |
| FP | |
| RC10-dT- | mCmUmAmAmAmAmCmAmCmCmAmCmAmUmGmAd |
| TegChol_dG | TdT-TegChol(SEQ ID NO: 148) |
| FP | |
| RC07- | mU#mA#mAmAmAmCmAmCmCmAmCmA#mU#mG |
| 2PS_dGFP | (SEQ ID NO: 149) |
| RC07- | mU#mA#mA#mAmAmCmAmCmCmAmC#mA#mU#mG |
| 3PS_dGFP | (SEQ ID NO: 150) |
| RC07- | mU#mA#mAmAmAmCmAmCmCmAmCmA#mU#mGdTd |
| dT_2PS- | T-TegChol(SEQ ID NO: 151) |
| TegChol_dG | |
| FP | |
| RC07-2PS- | mU#mA#mAmAmAmCmAmCmCmAmCmA#mU#mG- |
| TegChol_dG | TegChol(SEQ ID NO: 149) |
| FP | |

KEY:
rN = RNA,
mN = 2′-O-methyl RNA,
fN = 2′-fluoro RNA,
aN = 2′-NH₂ (2′-amino RNA),
sN = 4′-thio RNA,
dN = 2′-deoxy RNA, N = A, U, G, or C
N#N = phosphorothioate linkage

TABLE 4
Exemplary crRNAs.

| Name | Sequence |
|---|---|
| CO | mG#mA#mG#rArCrArArArUrCrArCrCrUrGrCrCr |
| | UrCrGrGrUrUrUrUrArGrArGrCrUrAmU#mGm#mC |
| | #mU(SEQ ID NO: 152) |
| C20 | mG#mA#mG#mAmCmAmAmAmUmCfAfCfCfUrG#rC#fC |
| | fUrC#mGmGrU#rU#rU#fUfAmGmAmGmCmUmAmU#mG |
| | #mC#mU(SEQ ID NO: 153) |
| C20- | mG#mA#mG#mAmCmAmAmAmUmCfAfCfCfUrG#rC#fC |
| GalNac | fUrC#mGmGrU#rU#rU#fUfAmGmAmGmCmUmAmU#mG |
| | #mC#mU-GalNac(SEQ ID NO: 153) |
| C20- | mG#mA#mG#mAmCmAmAmAmUmCfAfCfCfUrG#rC#fC |
| TegChol | fUrC#mGmGrU#rU#rU#fUfAmGmAmGmCmUmAmU#mG |
| | #mC#mU-TegChol(SEQ ID NO: 153) |
| C20- | mG#mA#mG#mAmCmAmAmAmUmCfAfCfCfUrG#rC#fC |
| Cy3_MCV1a | fUrC#mGmGrU#rU#rU#fUfAmGmAmGmCmUmAmU#mG |
| | #mC#mU-Cy3(SEQ ID NO: 153) |
| C20- | mG#mU#mC#mGmUmGmCmUmAmCfUfUfCfArU#rG#fU |
| GalNac_ | fGrG#mUmGrU#rU#rU#fUfAmGmAmGmCmUmAmU#m |
| dGFP | G#mC#mU-GalNac(SEQ ID NO: 154) |
| C20- | mG#mU#mC#mGmUmGmCmUmAmCfUfUfCfArU#rG#fU |
| DHA_dGFP | fGrG#mUmGrU#rU#rU#fUfAmGmAmGmCmUmAmU#m |
| | G#mC#mU-DHAv1(SEQ ID NO: 154) |
| C20-dT- | mG#mU#mC#mGmUmGmCmUmAmCfUfUfCfArU#rG#fU |
| DHA_dGFP | fGrG#mUmGrU#rU#rU#fUfAmGmAmGmCmUmAmU#m |
| | G#mC#mUdTdT-DHAv1(SEQ ID NO: 155) |
| C20- | mG#mU#mC#mGmUmGmCmUmAmCfUfUfCfArU#rG#fU |
| TegChol_ | fGrG#mUmGrU#rU#rU#fUfAmGmAmGmCmUmAmU#m |
| dGFP | G#mC#mU-TegChol(SEQ ID NO: 154) |
| C20-dT- | mG#mU#mC#mGmUmGmCmUmAmCfUfUfCfArU#rG#fU |
| TegChol_ | fGrG#mUmGrU#rU#rU#fUfAmGmAmGmCmUmAmU#m |
| dGFP | G#mC#mUdTdT-TegChol(SEQ ID NO: 155) |

KEY:
rN = RNA,
mN = 2′-O-methyl RNA,
fN = 2′-fluoro RNA,
aN = 2′-NH₂ (2′-amino RNA),
sN = 4′-thio RNA,
dN = 2′-deoxy RNA,
N = A, U, G, or C
N#N = phosphorothioate linkage

### Example 2—Protecting Oligos Enhance the Stability of crRNA Without Compromising Activity and Enhance the Potency of crRNA

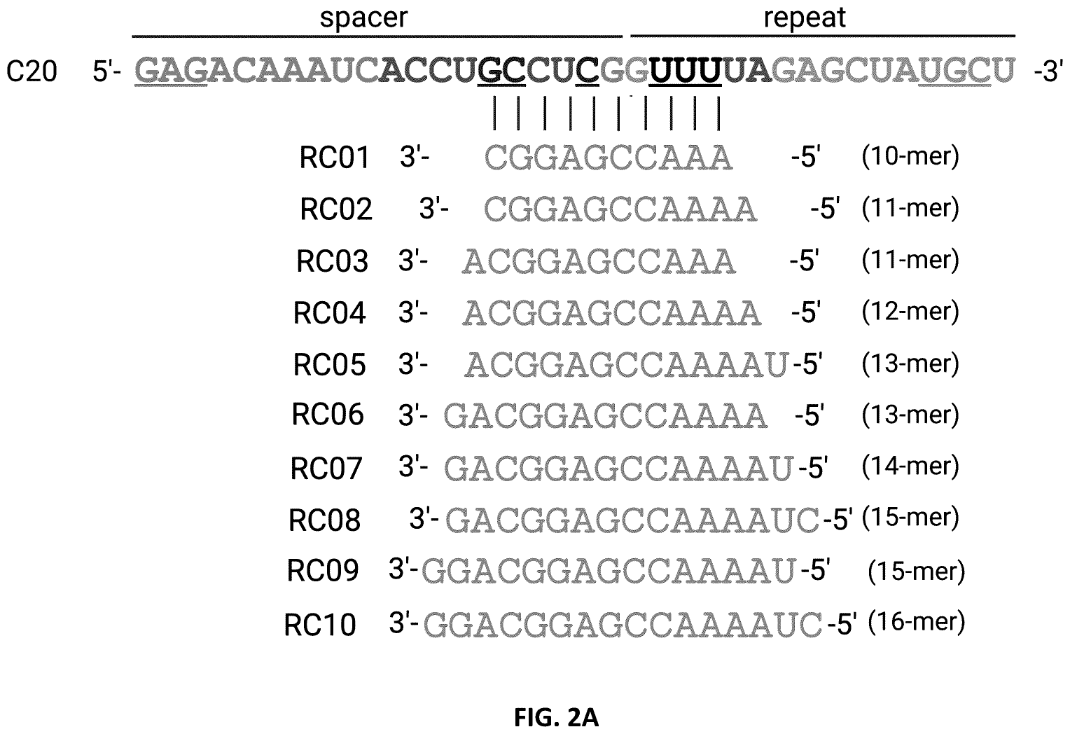

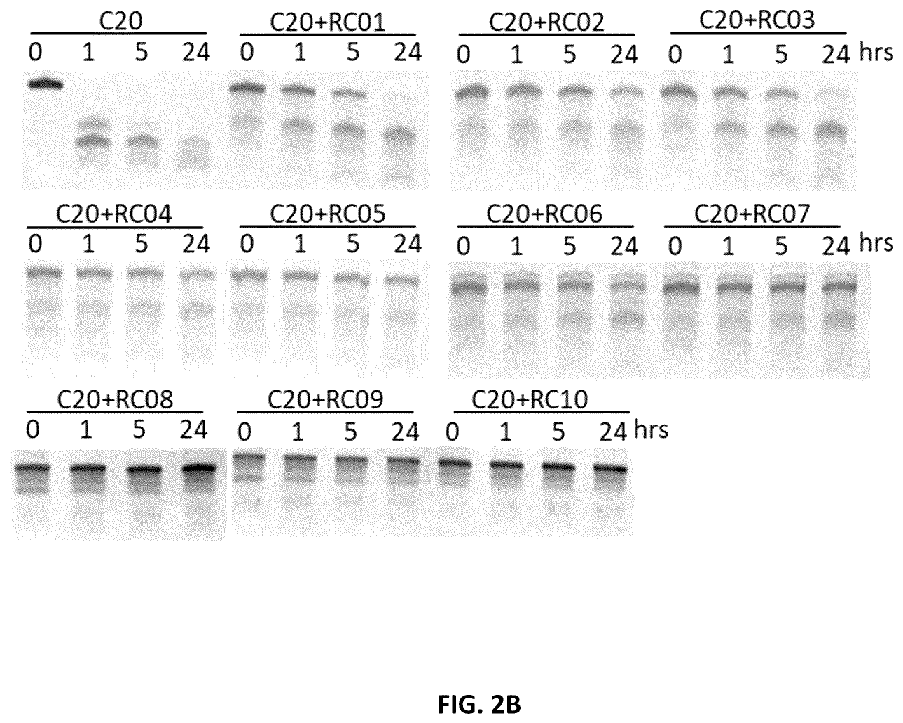

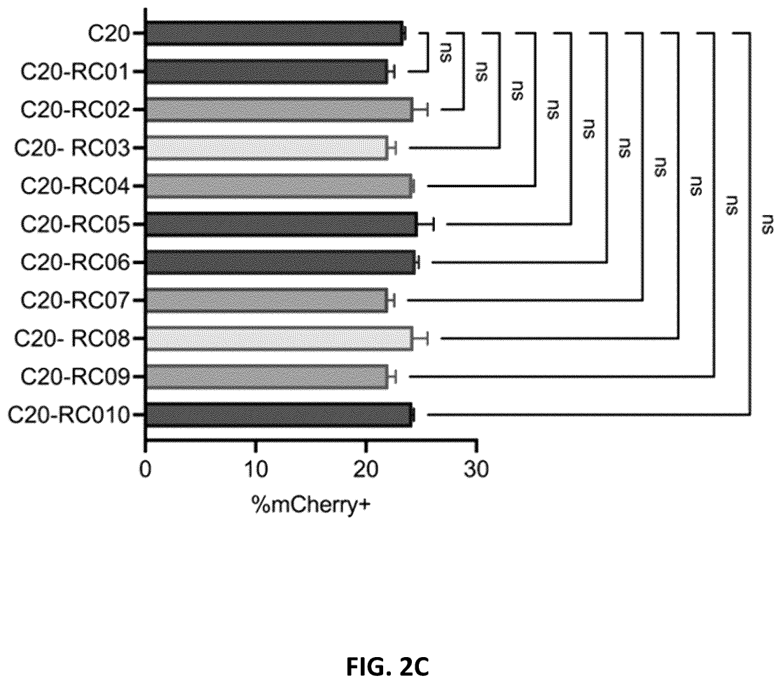

Inspired by the design of short DNA/RNA heteroduplex oligonucleotide (HDO) (Nishina et al., Nature Communications, 2015) and by the well-established observation that double-stranded RNAs are more resistant to nuclease degradation than single-stranded RNAs (Lam et al., Molecular Therapy Nucleic Acids, 2015), we sought to enhance the crRNA stability by designing short, fully chemically stabilized oligos that can protect the unmodified regions of C20 (FIG. 2A). We anticipated that the protecting oligo will be displaced by tracrRNA strand invasion during pairing of the crRNA repeat region with the tracrRNA anti-repeat region, thereby still allowing the crRNA/tracrRNA complex to form, as required for Cas9 editing. Formation of the crRNA/tracrRNA duplex would simultaneously induce dissociation of the protecting oligo. We found that when annealed to C20, oligos equal to or longer than 12 nt can protect C20 from degradation by nucleases in the FBS (FIG. 2B). By electroporating the C20-protecting oligo complexes into the stable cell line, we confirmed that protecting oligos do not interfere with SpyCas9 activity (FIG. 2C).

Protecting Oligos Enhance the Potency of crRNA

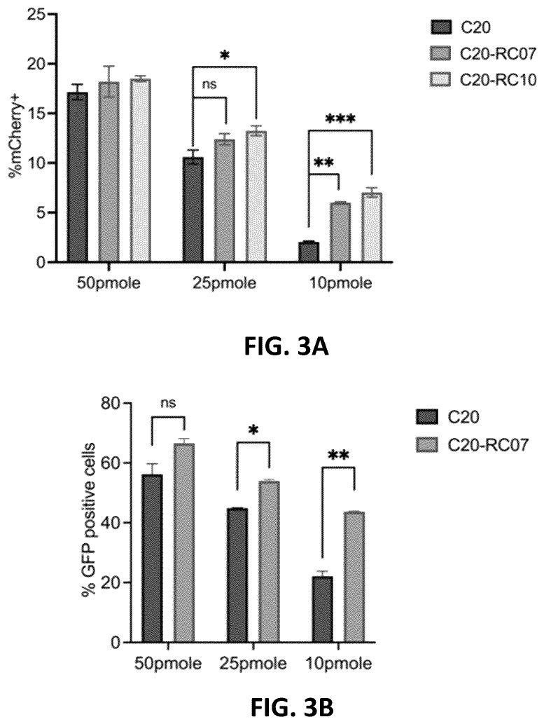

To test whether protecting oligos that enhanced C20 stability can also increase its potency, we electroporated the C20-protecting oligo complexes at non-saturating dosages. We found that protecting oligos significantly enhanced the potency of C20 (FIG. 3A and FIG. 3B).

### Example 3—Conjugation to Protecting Oligos Enhanced Cellular Uptake Without Interfering With crRNA Activity

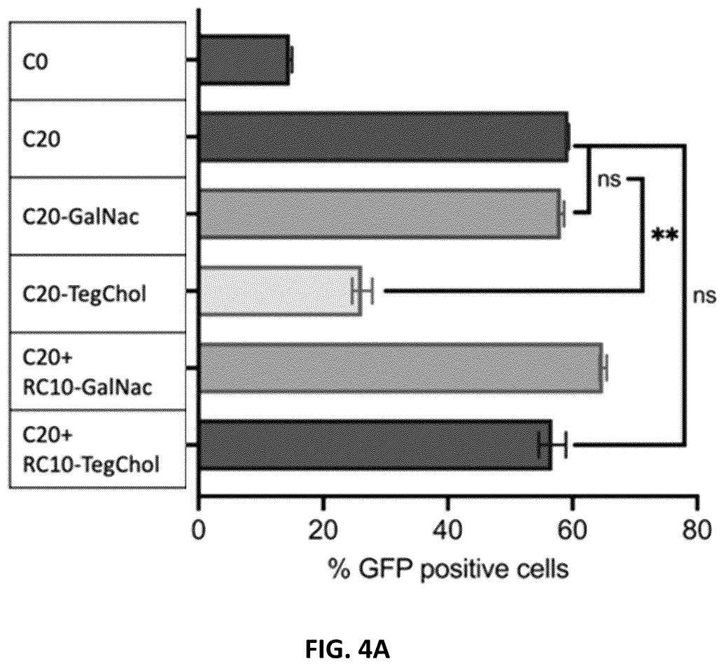

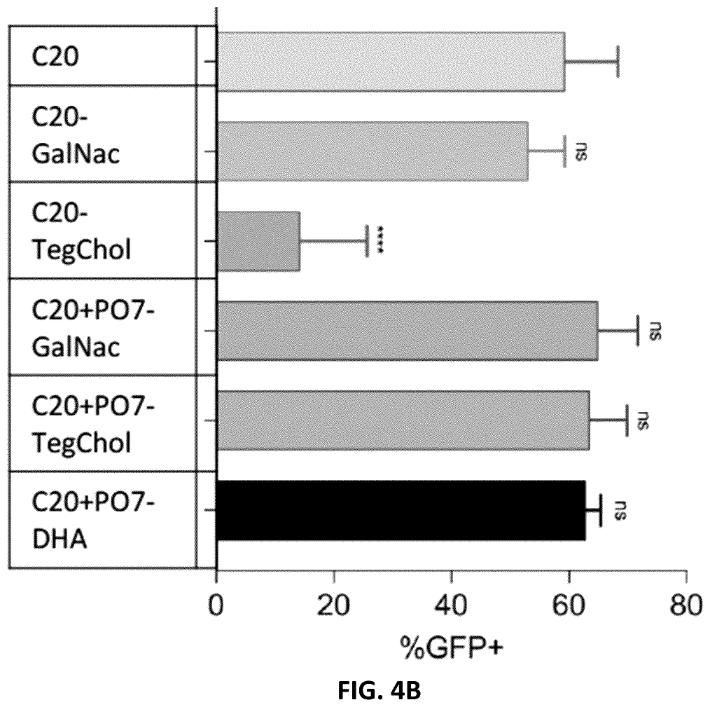

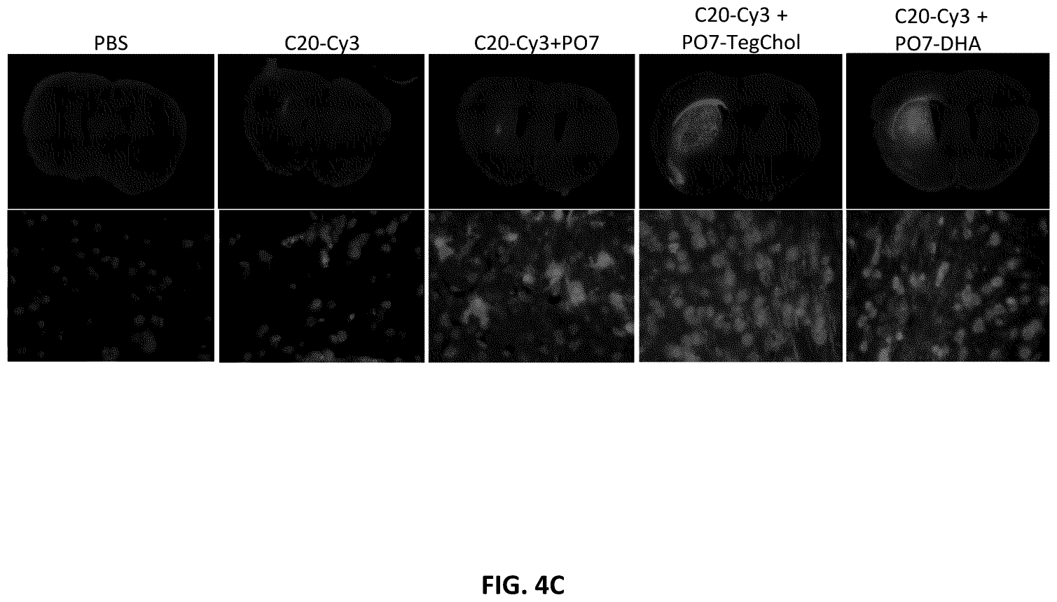

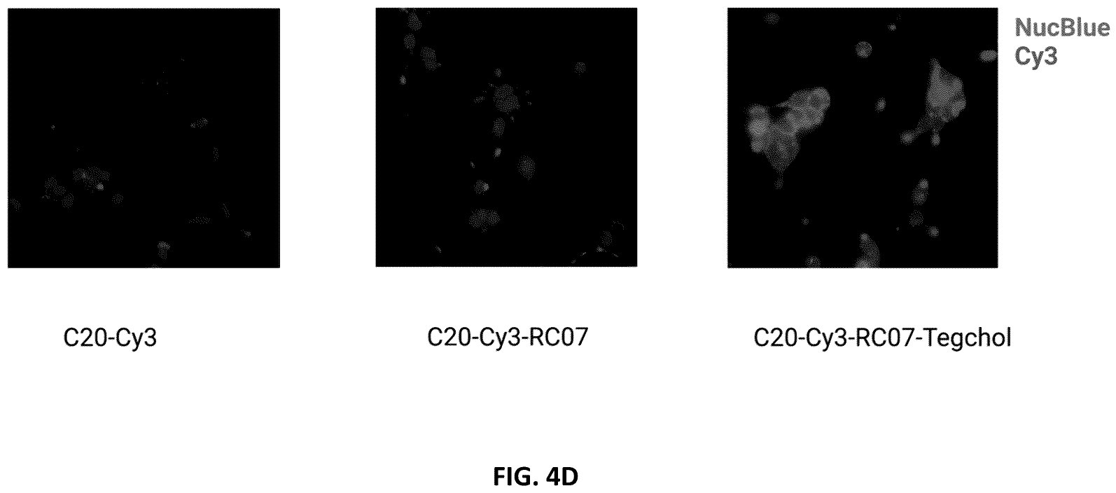

Addition of conjugations such as N-acetylgalactosamine (GalNac) and Cholesterol (TegChol) to therapeutic oligonucleotide is critical for achieving potent and targeted in vivo delivery. To test whether conjugations interfere with the crRNA activity, we synthesized both crRNA and protecting oligos with either GalNac or Cholesterol conjugation and tested the activity via electroporation in the stable cell line. We found that while directly conjugating of Cholesterol to C20 significantly compromised the activity, C20 with Cholesterol-conjugated protecting oligos did not show reduced activity (FIG. 4A). While C20 with cholesterol conjugation showed significantly reduced editing efficiency, conjugating cholesterol onto the protecting oligonucleotides did not interfere with the activity (FIG. 4B).

To directly visualize enhanced cellular uptake of C20 by conjugated protecting oligos in vivo, we delivered Cy3-labeled C20 with and without cholesterol- or DHA-conjugated protecting oligos in adult mouse brains by intrastriatal (IS) injection. We found that compared to unconjugated protecting oligos, both cholesterol- and DHA-conjugated protecting oligos significantly enhanced the in vivo distribution and cellular uptake of C20 (FIG. 4C). These data suggest that the protecting oligos allows various types of conjugations and can be used as a delivery vehicle to enhance cellular uptake of crRNA without interfering with the Cas9 activity. Moreover, we demonstrated that our engineered crRNA-protecting oligo complex can be efficiently taken up into cultured mammalian cells without transfection reagents (FIG. 4D).

### Example 4—Chemically Modified crRNAs With Varied Phosphorothioate Content

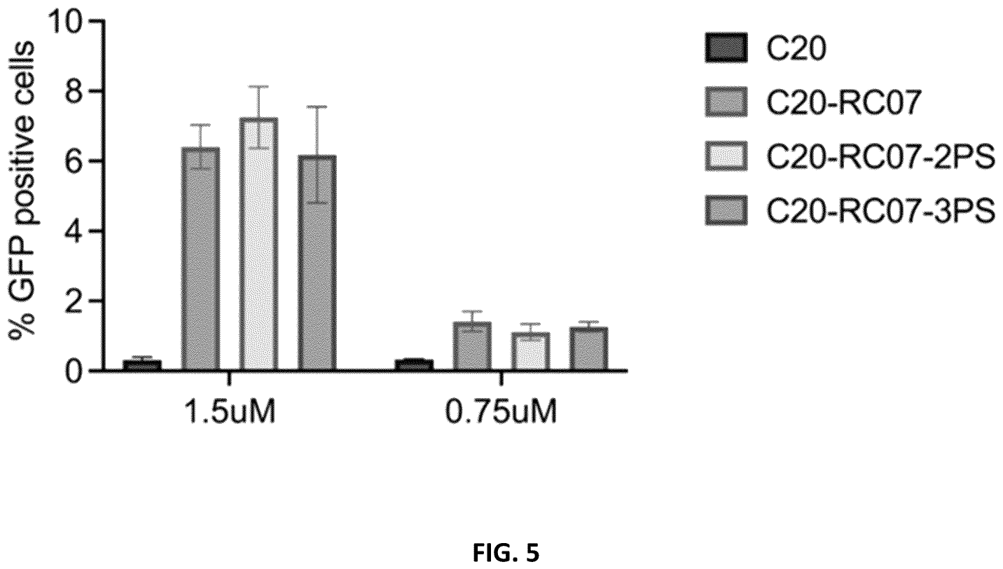

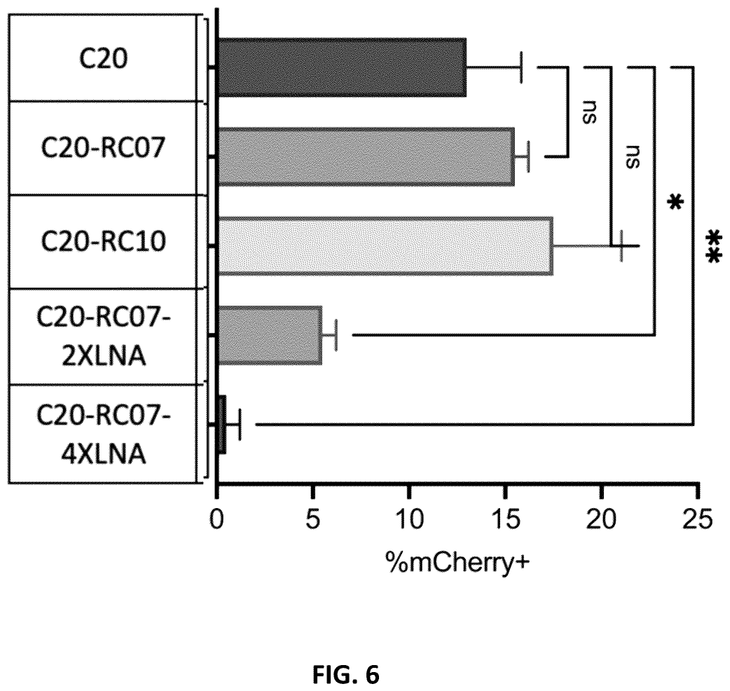

To test if the C20-protecting oligo complex can be taken up into cells passively (simply by adding it to the media, without electroporation or transfection), escape from endosomes, and support editing, we incubated the stable cell line shown in FIG. 1B with 1.5 μM C20-protecting oligo targeting the inactivated GFP reporter. Optimal duplex stability between protecting oligo and crRNA may be required for efficient editing (FIG. 6). We observed ˜7% editing efficiency (FIG. 5). Because cholesterol can be critical for siRNA uptake in cultured cells [Ly et al., Molecular Therapy Nucleic Acids 2020], further enhancement of editing efficiency can be achieved by using Cholesterol-conjugated protecting oligo as shown in FIG. 4A.

### Example 5—In Vivo Genome Editing in Adult Mouse CNS by Co-Delivery of AAV and Self-Delivering crRNA

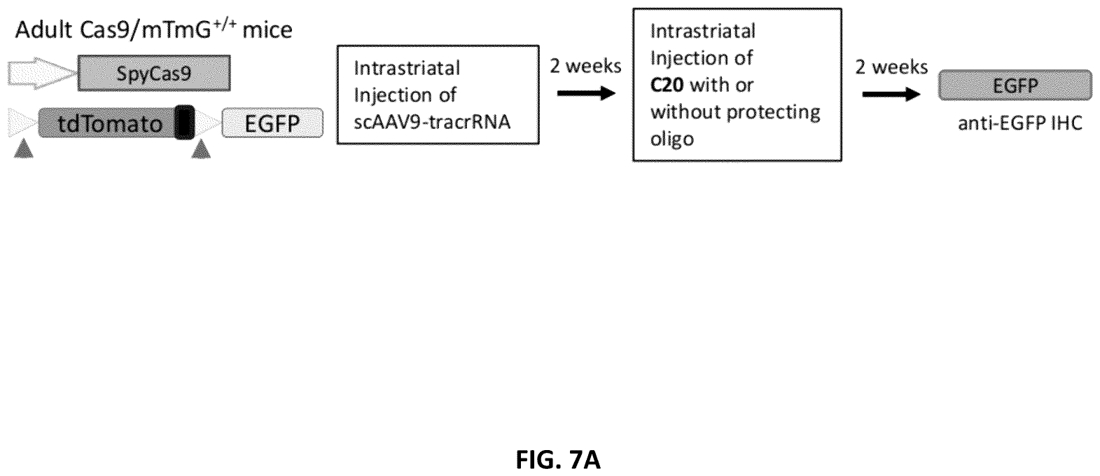

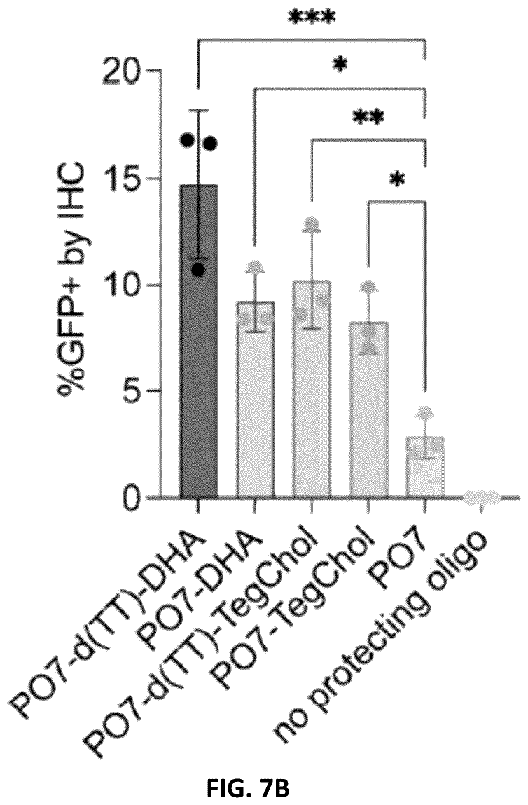

We tested whether the C20-protecting oligo complex can support genome editing by AAV co-delivery in vivo. We chose to test in a double transgenic reporter mouse model, Cas9/mTmG+/+. This mouse model was generated by crossing the homozygous Cas9+/+ mice68 with the homozygous mTmG+/+ reporter mice (Muzumdar et al. Genesis. 45: 593-605. 2007). The Cas9+/+ mouse model constitutively expresses SpyCas9, and the only transgene delivered from an AAV vector is the tracrRNA, which simplified the experimental process. The mTmG+/+ reporter consists of loxP sites on either side of a membrane-targeted tdTomato (mT) cassette, which constitutively expresses red fluorescent, and a downstream membrane-targeted EGFP (mG) that cannot be expressed until the mT cassette was deleted (FIG. 7A).

First, we injected scAAV9 that expresses tracrRNA into adult Cas9/mTmG+/+ mouse brain by IS injection. Two weeks later, we performed IS injection again to deliver naked C20, which targets the two loxP sites to generate block deletion of the mT cassette and allow the mG expression. We then detected GFP expression by performing Immunohistochemistry (IHC) on formalin-fixed mouse brain sections using anti-EGFP antibodies.

We chose to test both cholesterol and DHA conjugates, and both cleavable and stable linkers for the protecting oligos. We also included C20 with protecting oligos without any conjugation and C20 without protecting oligos as controls.

By IHC staining using an anti-EGFP antibody, we found that C20 without protecting oligos did not generate any editing in the brain. At the same time, we only observed a few EGFP-positive cells around the injection site with C20 and protecting oligos with no conjugation. In contrast, C20 with either DHA or cholesterol conjugation and with either cleavable or stable linker generated more efficient editing with broader distribution (FIG. 7B), which was consistent with the in vivo distribution results quantified in FIG. 4B. When quantifying the percentage of EGFP positive cells within 1 mm² around the site-of-injection, we found that the DHA conjugation performed better than the cholesterol conjugation and having a cleavable linker further improves the distribution and the editing efficiency (FIG. 7C).

### Example 6—Chemically Modified crRNAs With Varied Phosphorothioate Content

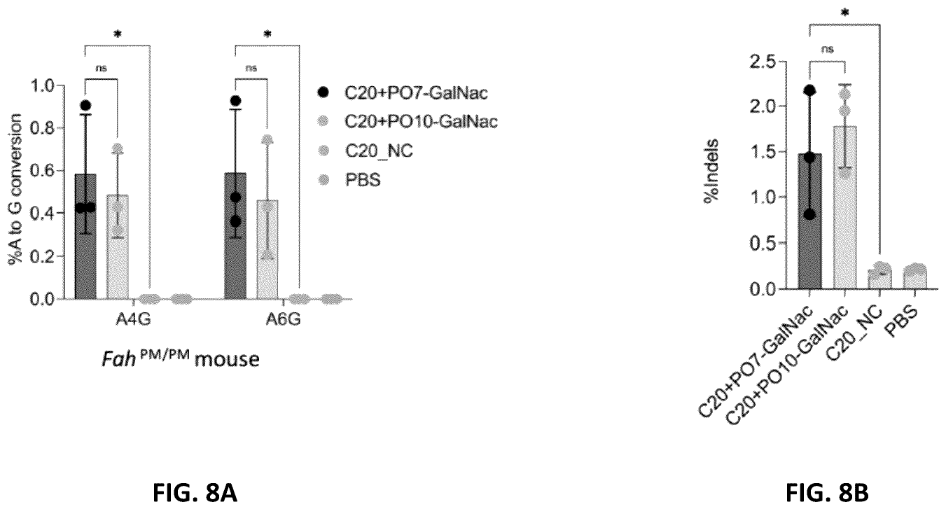

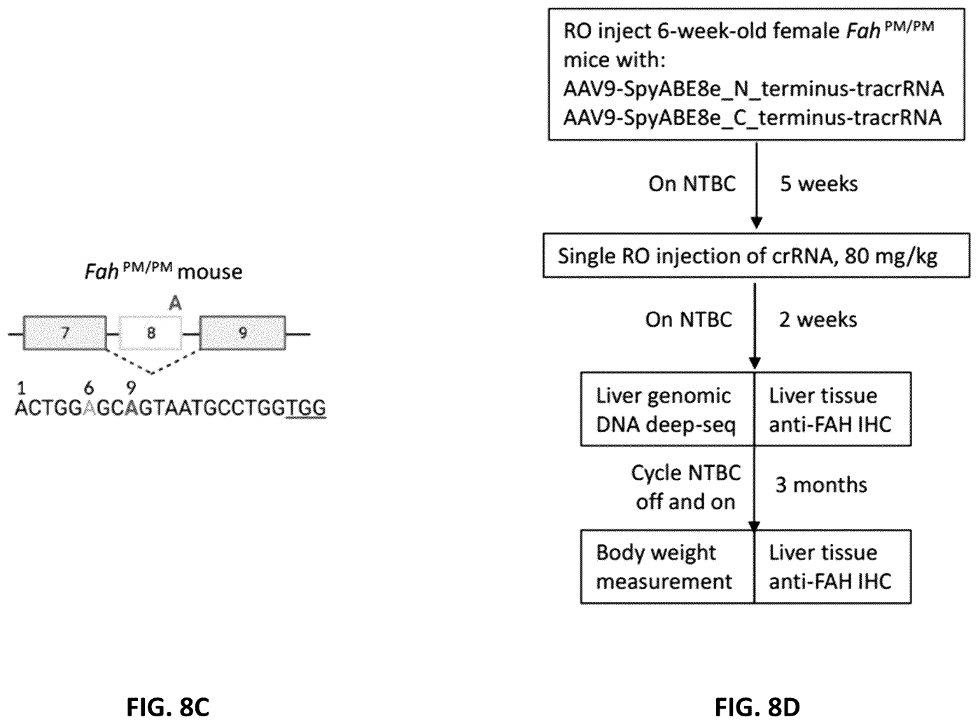

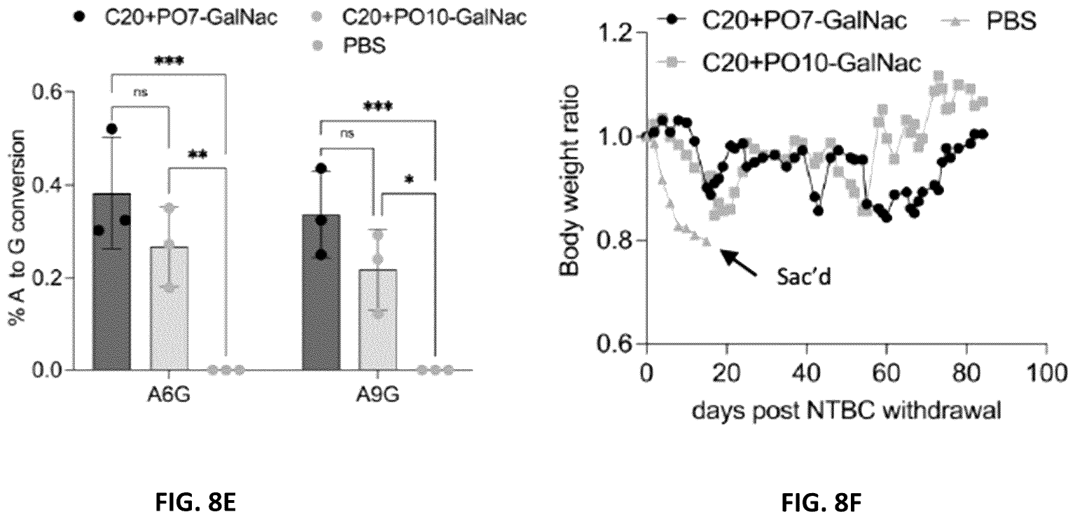

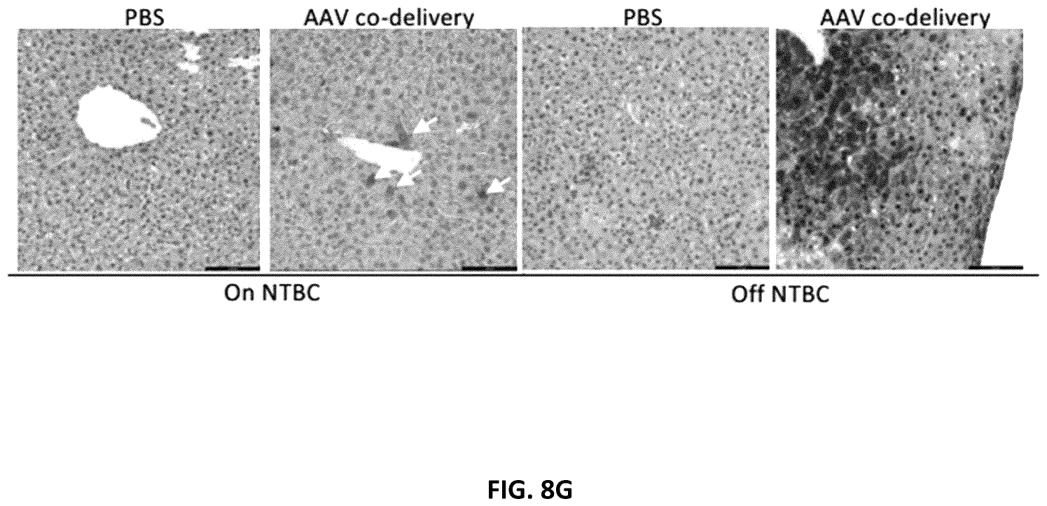

Using the adult wild-type B6 mice, we tested C20 targeting the mouse Pcsk9 gene with either 14 nucleotides or 16 nucleotides protecting oligo having a trivalent GalNac conjugation. We also included a non-targeting control C20, and PBS as controls. First, we injected adult mice with AAV to express either SpyCas9-ABE8e and tracrRNA (FIG. 8A), or SpyCas9 nuclease and tracrRNA (FIG. 8B) through retro-orbital (RO) injection and incubated five weeks to allow the effector protein to express and accumulate. We then injected C20 with and without GalNac-conjugated protecting oligo via a single RO injection at 80 mg/kg. The editing efficiency was assessed by extracting genomic DNA from the mouse liver tissue and performing targeted amplicon deep sequencing. We found low yet significant editing at the target genomic locus by C20 with both lengths of protecting oligo, while no editing was observed for the non-targeting control C20-injected mice (FIG. 8A and FIG. 8B).

Next, we tested whether our co-delivery approach can generate therapeutic-relevant editing by testing in the mouse model of HT1. The HT1 mouse model used in this study is the Fah^{PM/PM} mouse, which possesses a G-to-A point mutation in the last nucleotide of exon 8 of the Fah gene, which encodes fumarylacetoacetate hydrolase. This point mutation generates exon 8 skipping and causes FAH deficiency (FIG. 8C). Because FAH catalyzes one step of the tyrosine catabolic pathway, FAH deficiency leads to accumulations of toxic fumarylacetoacetate and succinyl acetoacetate, causing damage in multiple organs. The Fah^{PM/PM} mouse can be treated with 2-(2-nitro-4-trifluoromethylbenzoyl)-1,3-cyclohexanedione (NTBC), an inhibitor of an enzyme upstream within the tyrosine degradation pathway to prevent toxin accumulation. Without such treatment, the mice will rapidly lose weight and die. Previously, studies showed that the point mutation in the Fah^{PM/PM} mouse could be corrected by SpyCas9-ABE delivered by either lipid nanoparticle carrying mRNA and sgRNA or plasmid hydrodynamic tail-vein injection.

We first injected AAV to express SpyCas9-ABE8e and tracrRNA by RO injection. After five weeks, we performed RO injections of C20 with a previously validated spacer sequence targeting the point mutation in the Fah^{PM/PM} mouse, with either 14 nucleotides or 16 nucleotides trivalent GalNac-conjugated protecting oligos, at a single 80 mg/kg dose. The mice were kept on NTBC the whole time to maintain their body weight and allow time for the genome editing to occur. We then sacrificed three mice from each group and extracted liver genomic DNA to measure the editing efficiency by targeted amplicon deep sequencing, as well as detecting FAH-positive hepatocytes via IHC staining using anti-FAH antibodies. For the remaining mouse from each group, we cycled NTBC off and on (7-10 days off followed by 2 days on) for three months to allow FAH-positive hepatocytes to expand and monitor the mouse body weight to test if genome editing by our co-delivery approach can correct the phenotypes of this mouse model (FIG. 8D). Before NTBC withdrawal, we observed low yet significant on-target editing in the co-delivery-treated groups (FIG. 8E). After NTBC withdrawal, the PBS-injected mouse rapidly lost body weight and was humanely sacrificed after the body weight dropped below 80% and moribund. The mice treated with AAV-expressed SpyCas9-ABE8e and tracrRNA, and C20 with protecting oligos gradually gained body weight over time (FIG. 8F). The mice were then sacrificed after three months of NTBC cycles, and IHC staining was performed on liver sections using anti-FAH antibodies. We observed the expansion of FAH-positive hepatocytes in the liver tissue treated with AAV co-delivery (FIG. 8G). These data suggested that systemic delivery of C20 and GalNac-conjugated protecting oligos, with AAV-expressed effector protein and tracrRNA, can achieve in vivo genome editing in the liver and offer therapeutic benefits.

### Example 6—Improving the Editing Efficiency Through Optimization of the Dosing Regimen

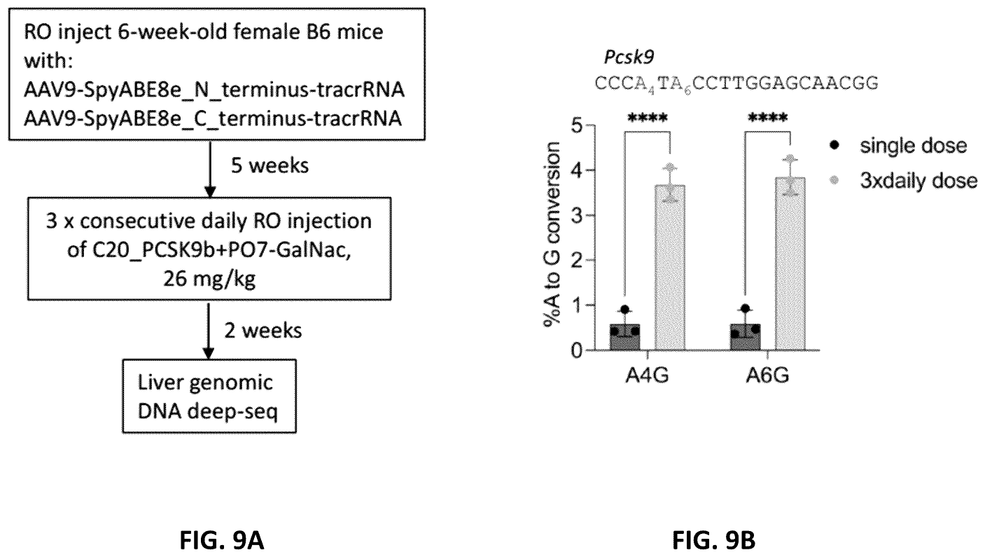

The editing efficiency we achieved in the liver, though significant, was low. We then sought to improve the efficiency by optimizing the dosing regimen. The asialoglycoprotein receptor (ASGPR), which is expressed on the surface of the hepatocytes and responsible for the uptake of GalNac-conjugated oligos, has a recycle time of around 10-15 minutes. Data from previous studies also showed that due to ASGPR saturation, the hepatocytes can only uptake limited amount of the GalNac-conjugated molecules after bolus intravenous injection.77 Our initial study injected a single dose of C20 with GalNac-conjugated protecting oligo at 80 mg/kg, far beyond the capacity of available ASGPR. We reasoned that instead of a single large dose, dividing the oligos into three consecutive daily RO injections with one-third of the previous dose (26 mg/kg) may be able to deliver more oligos into the hepatocyte, thus boosting the editing efficiency (FIG. 9A).

To test this idea, we first RO injected AAV to express SpyCas9-ABE8e and tracrRNA in the B6 mice. Then, after five weeks, we performed three consecutive daily RO injections of C20 targeting the Pcsk9 gene complexed with a 14 nt GalNac-conjugated protecting oligos at 26 mg/kg. We then harvested the mouse liver genomic DNA and performed targeted amplicon deep sequencing to measure the editing efficiency. We found that compared to a single dose of 80 mg/kg oligo, three consecutive daily doses at 26 mg/kg significantly improved the editing efficiency (FIG. 9B). In addition, we did not observe any sign of toxicity in the liver by redosing, and the oligo-treated mice were in good health, similar to the PBS-injected mice. These data indicate that repeat crRNA dosing is possible and can be well-tolerated, and optimizations of the dosing regimen will likely further increase the editing efficiency.

### Example 7—Improving the In Vivo Editing Efficiency Through Optimization of the Dosing Regimen

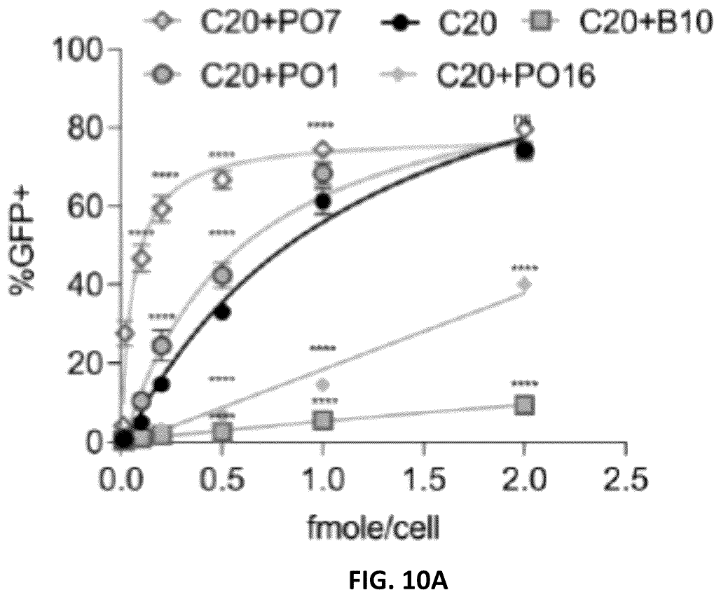

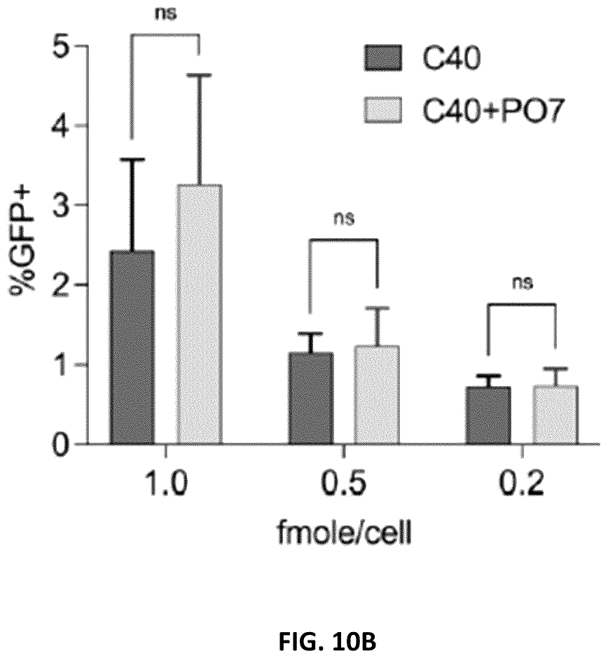

We then further explored the mechanisms of protecting oligo activity. We compared the activities of C20 with protecting oligonucleotides of different lengths or that anneal to different regions of C20 (FIG. 10). We found that protecting oligos as short as 10 nucleotides (P01) enhanced C20 potency, although to a lesser extent than the 14 nucleotides protecting oligo (P07), possibly because the lower melting temperature limited the degree of C20 protection. Conversely, we found that longer P.O.s (up to 22 nucleotides for P016, with a nucleotide sequence of 5′-GC ACAAAAACNNNN NNN-3′) (SEQ ID NO:156) reduced C20 potency, possibly due to the strong binding affinity that inhibited initial tracrRNA annealing, protecting oligo displacement, or both. Furthermore, the positions where the protecting oligo binds are also important (FIG. 10A). A 16 nt protecting oligo (B10 with a sequence of 5′-ACCAUAGCUCUAAAAC-3′) (SEQ ID NO:157) that leaves no toe-hold for initial tracrRNA annealing also leads to reduced activity, consistent with our hypothesis that the protecting oligo need to be displaced by tracrRNA before C20 can become active (FIG. 10A). Finally, we showed that the protecting oligo neither inhibited nor enhanced the potency of a fully modified and stabilized crRNA (C40), implying that the potency increase conferred by the protecting oligo resulted from C20 stabilization (FIG. 10B).

1. A protecting oligonucleotide comprising:
(a) a sequence that is complementary to a crRNA; and
(b) at least one chemically modified nucleotide,
wherein the protecting oligonucleotide is capable of binding the crRNA, and
wherein the protecting oligonucleotide confers nuclease resistance to the crRNA when bound.

2. The protecting oligonucleotide of claim 1, wherein the at least one chemically modified nucleotide comprises a modification of a ribose group, a phosphate group, a nucleobase, or a combination thereof, optionally wherein the modification of the ribose group is independently selected from the group consisting of 2′-O-methyl, 2′-fluoro, 2′-deoxy, 2′-O-(2-methoxyethyl) (MOE), 2′-NH₂(2′-amino), 4′-thio, a bicyclic nucleotide, a locked nucleic acid (LNA), a 2′-(S)-constrained ethyl (5-cEt), a constrained MOE, and a 2′-O,4′-C-aminomethylene bridged nucleic acid (2′,4′-BNA^{NC}).

3. (canceled)

4. The protecting oligonucleotide of claim 2, wherein at least 80% of the ribose groups are chemically modified.

5. The protecting oligonucleotide of claim 2, wherein at least 90% of the ribose groups are chemically modified.

6. The protecting oligonucleotide of claim 2, wherein 100% of the ribose groups are chemically modified.

7. (canceled)

8. The protecting oligonucleotide of claim 1, wherein the protecting oligonucleotide binds to the crRNA to form a duplex, optionally wherein the protecting oligonucleotide binds to a region of the crRNA that is not fully chemically modified.

9. The protecting oligonucleotide of claim 8, wherein the duplex has a melting temperature (Tm) of greater than 37° C. over the full length of the duplex.

10. The protecting oligonucleotide of claim 8, wherein the duplex has a melting temperature (Tm) of less than 37° C. over the region of complementarity comprising the guide sequence portion.

11. The protecting oligonucleotide of claim 1, wherein the binding of a tracrRNA to the guide sequence portion of the crRNA dissociates the protecting oligonucleotide from the crRNA.

12. The protecting oligonucleotide of claim 1, further comprising at least one moiety conjugated to the protecting oligonucleotide, optionally wherein the at least one moiety is selected from the group consisting of fatty acids, steroids, secosteroids, lipids, gangliosides analogs, nucleoside analogs, endocannabinoids, vitamins, receptor ligands, peptides, aptamers, and alkyl chains, or wherein the at least one moiety is selected from the group consisting of cholesterol, cholesterol-triethylene glycol (TEGChol), docosahexaenoic acid (DHA), docosanoic acid (DCA), lithocholic acid (LA), GalNAc, amphiphilic block copolymer (ABC), hydrophilic block copolymer (HBC), poloxamer, Cy5, and Cy3.

13-24. (canceled)

25. A double stranded oligonucleotide comprising:
(a) a crRNA comprising (i) a guide sequence portion capable of hybridizing to a target polynucleotide sequence, and (ii) a repeat sequence portion; and
(b) a protecting oligonucleotide that is complementary to the crRNA,
wherein the crRNA comprises at least 50% modified nucleotides, and
wherein the protecting oligonucleotide comprises at least one modified nucleotide.

26. The double stranded oligonucleotide of claim 25, wherein the at least one modified nucleotide in the protecting oligonucleotide or the modified nucleotides in the crRNA comprises a modification of a ribose group, a phosphate group, a nucleobase, or a combination thereof, optionally wherein the modification of the ribose group is independently selected from the group consisting of 2′-O-methyl, 2′-fluoro, 2′-deoxy, 2′-O-(2-methoxyethyl) (MOE), 2′-NH₂(2′-amino), 4′-thio, a bicyclic nucleotide, a locked nucleic acid (LNA), a 2′-(S)-constrained ethyl (5-cEt), a constrained MOE, and a 2′-O,4′-C-aminomethylene bridged nucleic acid (2′,4′-BNA^{NC}).

27-48. (canceled)

49. The double stranded oligonucleotide of claim 1, further comprising a tracrRNA comprising an anti-repeat nucleotide sequence that is complementary to the repeat sequence portion of the crRNA.

50. The double stranded oligonucleotide of claim 49, further comprising a nucleotide or non-nucleotide loop or linker linking the 3′ end of the crRNA portion to the 5′ end of the tracrRNA portion, optionally wherein:
the non-nucleotide linker comprises an ethylene glycol oligomer linker;
the nucleotide loop is chemically modified; and/or
the nucleotide loop comprises the nucleotide sequence of GAAA.

51. The protecting oligonucleotide or double stranded oligonucleotide of claim 1, comprising a crRNA portion modification pattern consisting of:
mN # mN # mN # mNmNmNmNmNmNmNfNfNfNfNrN # rN # fNfNrN # mNmGrU # rU # rU # fUfAmG mAmGmCmUmAmU # mG # mC # mU fSEQ ID NO:3) (crRNA 20); or
comprising a crRNA modification pattern selected from any of crRNA 1-134 of Table 2;
wherein rN=RNA, mN=2′-O-methyl RNA, fN=2′-fluoro RNA, N # N=phosphorothioate linkage, and N=any nucleotide.

52. The protecting oligonucleotide or double stranded oligonucleotide claim 1, wherein the protecting oligonucleotide comprises a modification pattern selected from the group consisting of:
mAmAmAmCmNmNmNmNmN (RC01);
mAmAmAmAmCmNmNmNmNmN (RC02);
mAmAmAmCmNmNmNmNmNmN (RC03);
mAmAmAmAmCmNmNmNmNmNmN (RC04);
mUmAmAmAmAmCmNmNmNmNmNmN (RC05);
mAmAmAmAmCmNmNmNmNmNmNmN (RC06);
mUmAmAmAmAmCmNmNmNmNmNmNmN (RC07);
mCmUmAmAmAmAmCmNmNmNmNmNmNmN (RC08);
mUmAmAmAmAmCmNmNmNmNmNmNmNmN (RC09);
mCmUmAmAmAmAmCmNmNmNmNmNmNmNmN (RC10);
mU#mA#mAmAmAmCmNmNmNmNmN#mN#mN (RC07-2PS);
and
mU#mA#mA#mAmAmCmNmNmNmN#mN#mN#mN (RC07-3PS),
wherein mN = 2′-O-methyl RNA,
N#N = phosphorothioate linkage,
and N = any nucleotide.

53-54. (canceled)

55. The protecting oligonucleotide or double stranded oligonucleotide of claim 52, wherein the guide RNA binds to a Cas9 nuclease selected from the group consisting of S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9), optionally wherein the Cas9 is a variant Cas9 with altered activity.

56-57. (canceled)

58. A method of editing a target region of a genome in a cell, comprising administering to said cell a genome editing system comprising:
the protecting oligonucleotide of claim 1;
one or more crRNAs of;
one or more tracrRNAs; and
an RNA-guided nuclease or a polynucleotide encoding an RNA-guided nuclease.

59. The method of claim 58, wherein:
the target gene is in a cell in an organism;
expression of the target gene is knocked out or knocked down; and/or
the RNA-guided nuclease or a polynucleotide encoding an RNA-guided nuclease and the one or more tracrRNAs are administered to the cell before the one or more crRNAs or the protecting oligonucleotide.

60-61. (canceled)

62. The oligonucleotide of claim 1, further comprising a linker linking the 3′ end of the crRNA portion to the 5′ end of the tracrRNA portion, optionally wherein:
the linker is cleavable, optionally wherein:
the linker is a cleavable d(TT)-PO-C7 linker.

63. (canceled)
